(19) United States
(12) Patent Application Publication
(10) Pub. No.: US 2024/0090962 A1
(43) Pub. Date: Mar. 21, 2024

(54) SYSTEMS AND METHODS FOR PROVIDING SYNTHETIC INDICATORS IN A USER INTERFACE FOR A ROBOT-ASSISTED SYSTEM

(71) Applicant: INTUITIVE SURGICAL OPERATIONS, INC., Sunnyvale, CA (US)

(72) Inventors: Brandon D. Itkowitz, San Jose, CA (US); Jason S. LaFrenais, San Jose, CA (US); Angel J. Perez Rosillo, San Francisco, CA (US)

(21) Appl. No.: 18/255,062

(22) PCT Filed: Nov. 29, 2021
(86) PCT No.: PCT/US2021/060917

§ 371 Date: May 30, 2023

Related U.S. Application Data
(60) Provisional application No. 63/119,549, filed on Nov. 30, 2020.

(51) Int. Cl. A61B 34/37; A61B 34/00; A61B 34/35; A61B 90/00

(52) U.S. Cl. CPC A61B 34/37; A61B 34/35; A61B 34/70; A61B 90/37; A61B 2090/363

(57) ABSTRACT

A medical system may comprise a display system and a control system. The control system may include a processing unit including one or more processors. The processing unit may be configured to display, on the display system, an image of a field, generated by an imaging component, of view of a surgical environment. The processing unit may also be configured to generate a three-dimensional synthetic indicator for a position of an instrument outside of the field of view of the surgical environment and display the three-dimensional synthetic indicator with the image of the field of view of the surgical environment.

### CROSS-REFERENCE TO RELATED APPLICATIONS

This application claims the benefit of U.S. Provisional Application 63/119,549 filed Nov. 30, 2020, which is incorporated by reference herein in its entirety.

### FIELD

The present disclosure is directed to medical procedures and methods for manipulating tissue during medical procedures. More particularly, the present disclosure is directed to systems and methods for providing depth-aware synthetic indicators, indicators for components outside of a field of view, and indicators for components occluded in the field of view.

### BACKGROUND

Minimally invasive medical techniques are intended to reduce the amount of extraneous tissue that is damaged during diagnostic or surgical procedures, thereby reducing patient recovery time, discomfort, and harmful side effects. Such minimally invasive techniques may be performed through natural orifices in a patient anatomy or through one or more surgical incisions. Through these natural orifices or incisions, clinicians may insert medical tools to reach a target tissue location. Minimally invasive medical tools include instruments such as therapeutic instruments, diagnostic instruments, and surgical instruments. Minimally invasive medical tools may also include imaging instruments such as endoscopic instruments that provide a user with a field of view within the patient anatomy.

Some minimally invasive medical tools may be robot-assisted including teleoperated, remotely operated, or otherwise computer-assisted. During a medical procedure, the clinician may be provided with a graphical user interface including an image of a three-dimensional field of view of the patient anatomy. To improve the clinician's experience and efficiency, various indicators may be needed to provide additional information about medical tools in the field of view, medical tools occluded in the field of view, and components outside of the field of view.

### SUMMARY

The embodiments of the invention are best summarized by the claims that follow the description.

In one example embodiment, a medical system may comprise a display system and a control system. The control system may include a processing unit including one or more processors. The processing unit may be configured to display, on the display system, an image of a field, generated by an imaging component, of view of a surgical environment. The processing unit may also be configured to generate a three-dimensional synthetic indicator for a position of an instrument outside of the field of view of the surgical environment and display the three-dimensional synthetic indicator with the image of the field of view of the surgical environment.

In another embodiment, a medical system may comprise a display system and an input system including a first pedal and a second pedal. The first pedal may have a spatial relationship to the second pedal. The medical system may also comprise a control system. The control system may include a processing unit including one or more processors. The processing unit may be configured to display, on the display system, an image of a field of view of a surgical environment. The image may be generated by an imaging component. The processing unit may also be configured to generate a first synthetic indicator indicating an engagement status of the first pedal, generate a second synthetic indicator indicating an engagement status of the second pedal, and display, on the display system, the first synthetic indicator relative to the second synthetic indicator based on the spatial relationship with the image of the field of view of the surgical environment.

In another embodiment, a medical system may comprise a display system and an input system including a first pedal and a second pedal. The first pedal may have a spatial relationship to the second pedal. The medical system may also comprise a control system. The control system may include a processing unit including one or more processors. The processing unit may be configured to display, on the display system, an image of a field of view of a surgical environment. The image may be generated by an imaging component. The processing unit may also be configured to generate a first synthetic indicator associated with an instrument in the surgical environment, generate a depth mapping including the first synthetic indicator and a structure in the field of view, and determine, from the depth mapping, an occluded portion of the first synthetic indicator occluded by the structure. The processing unit may also be configured to display, on the display system, the first synthetic indicator. The occluded portion of the first synthetic indicator may have a differentiated graphic appearance from a non-occluded portion of the first synthetic indicator.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1A is a schematic view of a medical system, in accordance with an embodiment.

FIG. 1B is a perspective view of an assembly, in accordance with an embodiment.

FIG. 1C is a perspective view of a surgeon's control console for a medical system, in accordance with an embodiment.

FIGS. 2A, 2B, 2C, and 2D illustrate a graphical user interface with synthetic indicators pointing in the direction of offscreen tools, according to some embodiments.

FIGS. 3A, 3B, 3C, 3D, and 3E illustrate a synthetic indicator in various three-dimensional orientations pointing to different locations of a medical tool, according to some embodiments.

FIG. 3F illustrates a top-view of the stereoscopic viewing frustum of an endoscope, according to some embodiments.

FIGS. 3G-3J provide a progression of images depicting a modulation of the length of the synthetic indicator, according to some embodiments.

FIG. 4 is a top view of an input control apparatus that includes a food pedal panel and a sensor system, according to some embodiments.

FIGS. 5A, 5B, 5C, and 5D illustrate a graphical user interface with synthetic indicators providing status information about foot pedals associated with onscreen tools, according to some embodiments.

FIGS. 6A, 6B, 6C, and 6D illustrate a graphical user interface with synthetic indicators providing status information about foot pedals associated with onscreen tools, according to some embodiments.

FIGS. 7A, 7B, 7C, and 7D illustrate a graphical user interface with synthetic indicators that may conditionally move to stay visible as the components or the endoscope generating the field of view are moved, according to some embodiments.

FIG. 8 illustrates an endoscope 550 extending into a patient anatomy to visualize synthetic indicators on a medical tool, according to some embodiments.

FIGS. 9A and 9B illustrate a graphical user interface with synthetic indicators that remain visible when occluded, according to some embodiments.

FIGS. 10A, 10B, and 10C illustrate a graphical user interface with synthetic indicators having occluded portions, according to some embodiments.

FIGS. 11A, 11B, 11C, and 11D illustrate a graphical user interface with a synthetic indicator for guiding a tool change, according to some embodiments.

FIG. 12 is a flowchart describing a method for displaying a synthetic indicator to point toward and offscreen tool, according to some embodiments.

FIG. 13 is a flowchart describing a method for displaying a synthetic indicator indicate a status of a foot pedal engagement, according to some embodiments.

FIG. 14 is a flowchart describing a method for displaying a synthetic indicator that is at least partially occluded by a structure in the field of view, according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In robot-assisted medical procedures, endoscopic images of the surgical environment may provide a clinician with a field of view of the patient anatomy and any medical tools located in the patient anatomy. Augmenting the endoscopic images with various indicators may allow the clinician to access information while maintaining the field of view. Such indicators may include depth-aware graphical indicators, indicators for components outside of a field of view, and indicators for components occluded in the field of view.

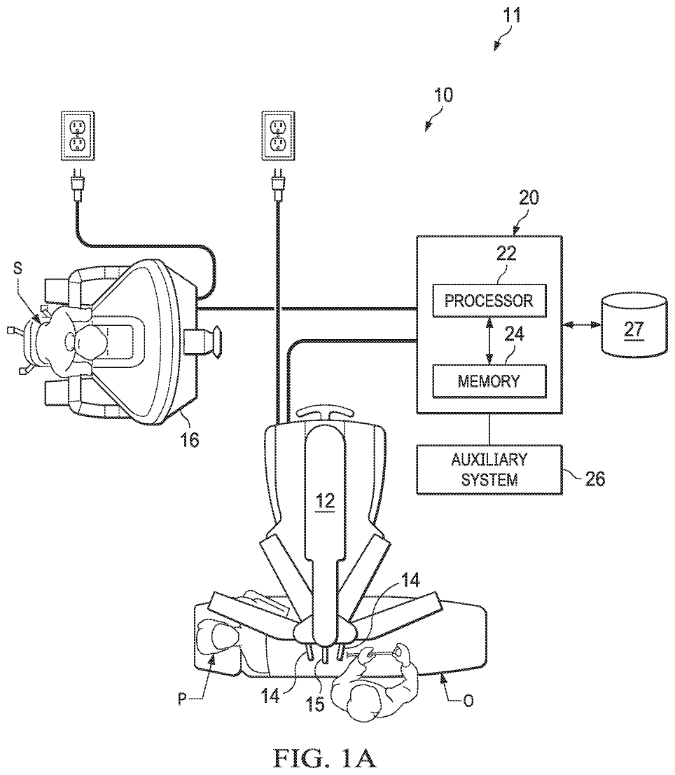

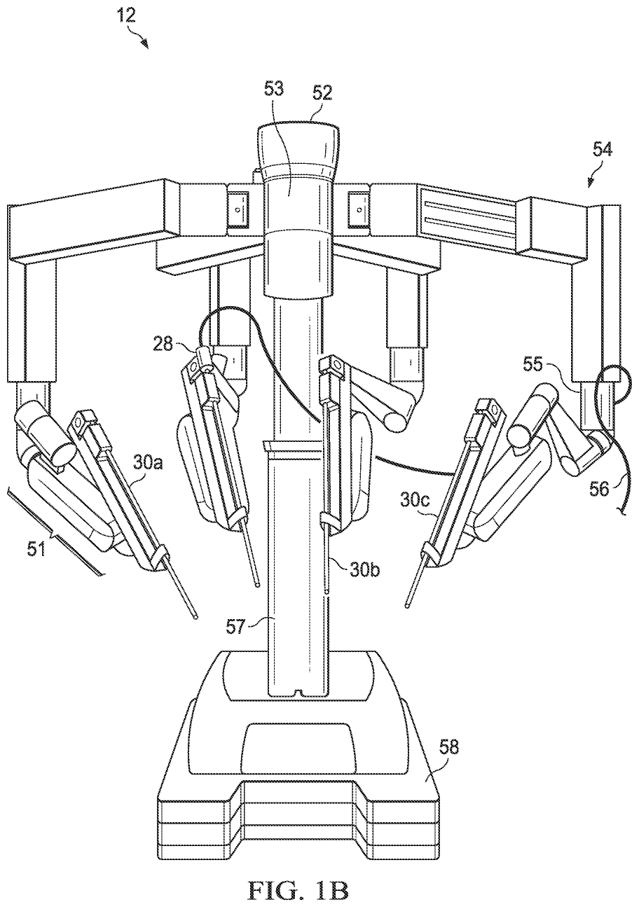

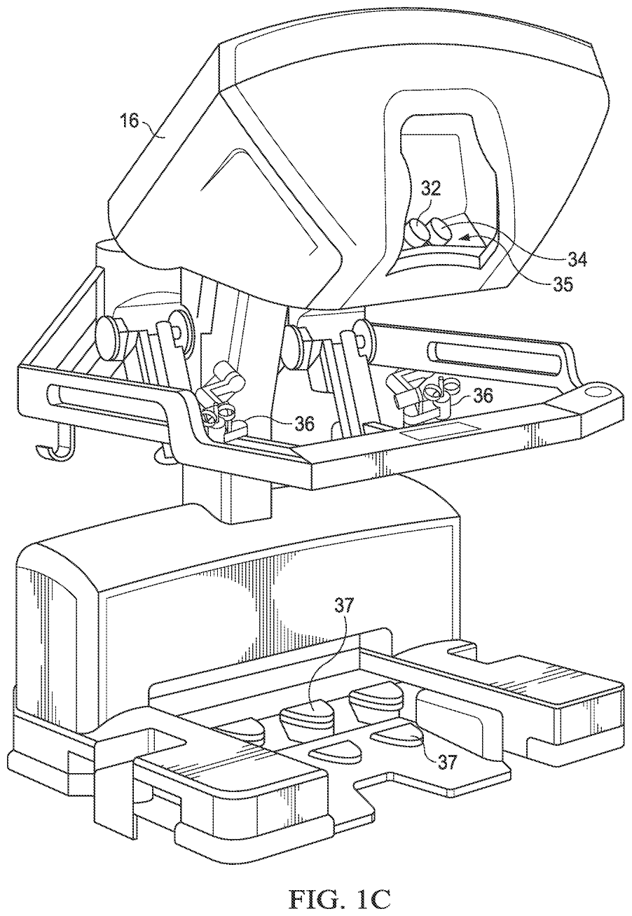

FIGS. 1A, 1B, and 1C together provide an overview of a medical system 10 that may be used in, for example, medical procedures including diagnostic, therapeutic, or surgical procedures. The medical system 10 is located in a medical environment 11. The medical environment 11 is depicted as an operating room in FIG. 1A. In other embodiments, the medical environment 11 may be an emergency room, a medical training environment, a medical laboratory, or some other type of environment in which any number of medical procedures or medical training procedures may take place. In still other embodiments, the medical environment 11 may include an operating room and a control area located outside of the operating room.

In one or more embodiments, the medical system 10 may be a robot-assisted medical system that is under the teleoperational control of a surgeon. In alternative embodiments, the medical system 10 may be under the partial control of a computer programmed to perform the medical procedure or sub-procedure. In still other alternative embodiments, the medical system 10 may be a fully automated medical system that is under the full control of a computer programmed to perform the medical procedure or sub-procedure with the medical system 10. One example of the medical system 10 that may be used to implement the systems and techniques described in this disclosure is the da Vinci® Surgical System manufactured by Intuitive Surgical, Inc. of Sunnyvale, California.

As shown in FIG. 1A, the medical system 10 generally includes an assembly 12, which may be mounted to or positioned near an operating table O on which a patient P is positioned. The assembly 12 may be referred to as a patient side cart, a surgical cart, or a surgical robot. In one or more embodiments, the assembly 12 may be a teleoperational assembly. The teleoperational assembly may be referred to as, for example, a teleoperational arm cart. A medical instrument system 14 and an endoscopic imaging system 15 are operably coupled to the assembly 12. An operator input system 16 allows a surgeon S or other type of clinician to view images of or representing the surgical site and to control the operation of the medical instrument system 14 and/or the endoscopic imaging system 15.

The medical instrument system 14 may comprise one or more medical instruments. In embodiments in which the medical instrument system 14 comprises a plurality of medical instruments, the plurality of medical instruments may include multiple of the same medical instrument and/or multiple different medical instruments. Similarly, the endoscopic imaging system 15 may comprise one or more endoscopes. In the case of a plurality of endoscopes, the plurality of endoscopes may include multiple of the same endoscope and/or multiple different endoscopes.

The operator input system 16 may be located at a surgeon's control console, which may be located in the same room as operating table O. In some embodiments, the surgeon S and the operator input system 16 may be located in a different room or a completely different building from the patient P. The operator input system 16 generally includes one or more control device(s) for controlling the medical instrument system 14. The control device(s) may include one or more of any number of a variety of input devices, such as hand grips, joysticks, trackballs, data gloves, trigger-guns, foot pedals, hand-operated controllers, voice recognition devices, touch screens, body motion or presence sensors, and other types of input devices.

In some embodiments, the control device(s) will be provided with the same degrees of freedom as the medical instrument(s) of the medical instrument system 14 to provide the surgeon with telepresence, which is the perception that the control device(s) are integral with the instruments so that the surgeon has a strong sense of directly controlling instruments as if present at the surgical site. In other embodiments, the control device(s) may have more or fewer degrees of freedom than the associated medical instruments and still provide the surgeon with telepresence. In some embodiments, the control device(s) are manual input devices that move with six degrees of freedom, and which may also include an actuatable handle for actuating instruments (for example, for closing grasping jaw end effectors, applying an electrical potential to an electrode, delivering a medicinal treatment, and actuating other types of instruments).

The assembly 12 supports and manipulates the medical instrument system 14 while the surgeon S views the surgical site through the operator input system 16. An image of the surgical site may be obtained by the endoscopic imaging system 15, which may be manipulated by the assembly 12. The assembly 12 may comprise endoscopic imaging systems 15 and may similarly comprise multiple medical instrument systems 14 as well. The number of medical instrument systems 14 used at one time will generally depend on the diagnostic or surgical procedure to be performed and on space constraints within the operating room, among other factors. The assembly 12 may include a kinematic structure of one or more non-servo controlled links (e.g., one or more links that may be manually positioned and locked in place, generally referred to as a set-up structure) and a manipulator. When the manipulator takes the form of a teleoperational manipulator, the assembly 12 is a teleoperational assembly. The assembly 12 includes a plurality of motors that drive inputs on the medical instrument system 14. In an embodiment, these motors move in response to commands from a control system (e.g., control system 20). The motors include drive systems which when coupled to the medical instrument system 14 may advance a medical instrument into a naturally or surgically created anatomical orifice. Other motorized drive systems may move the distal end of said medical instrument in multiple degrees of freedom, which may include three degrees of linear motion (e.g., linear motion along the X, Y, Z Cartesian axes) and three degrees of rotational motion (e.g., rotation about the X, Y, Z Cartesian axes). Additionally, the motors may be used to actuate an articulable end effector of the medical instrument for grasping tissue in the jaws of a biopsy device or the like. Medical instruments of the medical instrument system 14 may include end effectors having a single working member such as a scalpel, a blunt blade, an optical fiber, or an electrode. Other end effectors may include, for example, forceps, graspers, scissors, or clip appliers.

The medical system 10 also includes a control system 20. The control system 20 includes at least one memory 24 and at least one processor 22 for effecting control between the medical instrument system 14, the operator input system 16, and other auxiliary systems 26 which may include, for example, imaging systems, audio systems, fluid delivery systems, display systems, illumination systems, steering control systems, irrigation systems, and/or suction systems. A clinician may circulate within the medical environment 11 and may access, for example, the assembly 12 during a set up procedure or view a display of the auxiliary system 26 from the patient bedside.

Though depicted as being external to the assembly 12 in FIG. 1A, the control system 20 may, in some embodiments, be contained wholly within the assembly 12. The control system 20 also includes programmed instructions (e.g., stored on a non-transitory, computer-readable medium) to implement some or all of the methods described in accordance with aspects disclosed herein. While the control system 20 is shown as a single block in the simplified schematic of FIG. 1A, the control system 20 may include two or more data processing circuits with one portion of the processing optionally being performed on or adjacent the assembly 12, another portion of the processing being performed at the operator input system 16, and the like.

Any of a wide variety of centralized or distributed data processing architectures may be employed. Similarly, the programmed instructions may be implemented as a number of separate programs or subroutines, or they may be integrated into a number of other aspects of the systems described herein, including teleoperational systems. In one embodiment, the control system 20 supports wireless communication protocols such as Bluetooth, IrDA, HomeRF, IEEE 802.11, DECT, and Wireless Telemetry.

The control system 20 is in communication with a database 27 which may store one or more clinician profiles, a list of patients and patient profiles, a list of procedures to be performed on said patients, a list of clinicians scheduled to perform said procedures, other information, or combinations thereof. A clinician profile may comprise information about a clinician, including how long the clinician has worked in the medical field, the level of education attained by the clinician, the level of experience the clinician has with the medical system 10 (or similar systems), or any combination thereof.

The database 27 may be stored in the memory 24 and may be dynamically updated. Additionally or alternatively, the database 27 may be stored on a device such as a server or a portable storage device that is accessible by the control system 20 via an internal network (e.g., a secured network of a medical facility or a teleoperational system provider) or an external network (e.g. the Internet). The database 27 may be distributed throughout two or more locations. For example, the database 27 may be present on multiple devices which may include the devices of different entities and/or a cloud server. Additionally or alternatively, the database 27 may be stored on a portable user-assigned device such as a computer, a mobile device, a smart phone, a laptop, an electronic badge, a tablet, a pager, and other similar user devices.

In some embodiments, control system 20 may include one or more servo controllers that receive force and/or torque feedback from the medical instrument system 14. Responsive to the feedback, the servo controllers transmit signals to the operator input system 16. The servo controller(s) may also transmit signals instructing assembly 12 to move the medical instrument system(s) 14 and/or endoscopic imaging system 15 which extend into an internal surgical site within the patient body via openings in the body. Any suitable conventional or specialized servo controller may be used. A servo controller may be separate from, or integrated with, assembly 12. In some embodiments, the servo controller and assembly 12 are provided as part of a teleoperational arm cart positioned adjacent to the patient's body.

The control system 20 can be coupled with the endoscopic imaging system 15 and can include a processor to process captured images for subsequent display, such as to a surgeon on the surgeon's control console, or on another suitable display located locally and/or remotely. For example, where a stereoscopic endoscope is used, the control system 20 can process the captured images to present the surgeon with coordinated stereo images of the surgical site. Such coordination can include alignment between the opposing images and can include adjusting the stereo working distance of the stereoscopic endoscope.

In alternative embodiments, the medical system 10 may include more than one assembly 12 and/or more than one operator input system 16. The exact number of assemblies 12 will depend on the surgical procedure and the space constraints within the operating room, among other factors. The operator input systems 16 may be collocated or they may be positioned in separate locations. Multiple operator input systems 16 allow more than one operator to control one or more assemblies 12 in various combinations. The medical system 10 may also be used to train and rehearse medical procedures.

FIG. 1B is a perspective view of one embodiment of an assembly 12 which may be referred to as a patient side cart, surgical cart, teleoperational arm cart, or surgical robot. The assembly 12 shown provides for the manipulation of three surgical tools 30a, 30b, and 30c (e.g., medical instrument systems 14) and an imaging device 28 (e.g., endoscopic imaging system 15), such as a stereoscopic endoscope used for the capture of images of the site of the procedure. The imaging device may transmit signals over a cable 56 to the control system 20. Manipulation is provided by teleoperative mechanisms having a number of joints. The imaging device 28 and the surgical tools 30a-c can be positioned and manipulated through incisions in the patient so that a kinematic remote center is maintained at the incision to minimize the size of the incision. Images of the surgical site can include images of the distal ends of the surgical tools 30a-c when they are positioned within the field-of-view of the imaging device 28.

The assembly 12 includes a drivable base 58. The drivable base 58 is connected to a telescoping column 57, which allows for adjustment of the height of arms 54. The arms 54 may include a rotating joint 55 that both rotates and moves up and down. Each of the arms 54 may be connected to an orienting platform 53. The arms 54 may be labeled to facilitate trouble shooting. For example, each of the arms 54 may be emblazoned with a different number, letter, symbol, other identifier, or combinations thereof. The orienting platform 53 may be capable of 360 degrees of rotation. The assembly 12 may also include a telescoping horizontal cantilever 52 for moving the orienting platform 53 in a horizontal direction.

In the present example, each of the arms 54 connects to a manipulator arm 51. The manipulator arms 51 may connect directly to a medical instrument, e.g., one of the surgical tools 30a-c. The manipulator arms 51 may be teleoperable. In some examples, the arms 54 connecting to the orienting platform 53 may not be teleoperable. Rather, such arms 54 may be positioned as desired before the surgeon S begins operation with the teleoperative components. Throughout a surgical procedure, medical instruments may be removed and replaced with other instruments such that instrument to arm associations may change during the procedure.

Endoscopic imaging systems (e.g., endoscopic imaging system 15 and imaging device 28) may be provided in a variety of configurations including rigid or flexible endoscopes. Rigid endoscopes include a rigid tube housing a relay lens system for transmitting an image from a distal end to a proximal end of the endoscope. Flexible endoscopes transmit images using one or more flexible optical fibers. Digital image-based endoscopes have a “chip on the tip” design in which a distal digital sensor such as a one or more charge-coupled device (CCD) or a complementary metal oxide semiconductor (CMOS) device store image data. Endoscopic imaging systems may provide two- or three-dimensional images to the viewer. Two-dimensional images may provide limited depth perception. Three-dimensional stereo endoscopic images may provide the viewer with more accurate depth perception. Stereo endoscopic instruments employ stereo cameras to capture stereo images of the patient anatomy. An endoscopic instrument may be a fully sterilizable assembly with the endoscope cable, handle and shaft all rigidly coupled and hermetically sealed.

FIG. 1C is a perspective view of an embodiment of the operator input system 16 at the surgeon's control console. The operator input system 16 includes a left eye display 32 and a right eye display 34 for presenting the surgeon S with a coordinated stereo view of the surgical environment that enables depth perception. The left and right eye displays 32, 32 may be components of a display system 35. In other embodiments, the display system 35 may include one or more other types of displays. The display system 35 may present images captured, for example, by the imaging system 15 to display the endoscopic field of view to the surgeon. The endoscopic field of view may be augmented by virtual or synthetic menus, indicators, and/or other graphical or textual information to provide additional information to the viewer.

The operator input system 16 further includes one or more input control devices 36, which in turn cause the assembly 12 to manipulate one or more instruments of the endoscopic imaging system 15 and/or medical instrument system 14. The input control devices 36 can provide the same degrees of freedom as their associated instruments to provide the surgeon S with telepresence, or the perception that the input control devices 36 are integral with said instruments so that the surgeon has a strong sense of directly controlling the instruments. To this end, position, force, and tactile feedback sensors (not shown) may be employed to transmit position, force, and tactile sensations from the medical instruments, e.g., surgical tools 30a-c, or imaging device 28, back to the surgeon's hands through the input control devices 36. Input control devices 37 are foot pedals that receive input from a user's foot. Aspects of the operator input system 16, the assembly 12, and the auxiliary systems 26 may be adjustable and customizable to meet the physical needs, skill level, or preferences of the surgeon S.

During a medical procedure performed using the medical system 10, the surgeon S or another clinician may need to access medical tools in the patient anatomy that are outside of the field of view of the imaging system 15, may need to engage foot pedals activate medical tools or perform other system functions, and/or may need to identify tools that are occluded in the field of view. Further, with a stereoscopic field of view, it may be desirable that synthetic elements presented with the field of view are displayed at depths that correspond with the tissue or components indicated by the synthetic elements. Thus, the synthetic elements may appear to be attached to the components in the field of view rather than floating in front of the field of view. The various embodiments described below provide methods and systems that allow the surgeon S to view depth-aware graphical indicators, indicators for components outside of a field of view, and indicators for components occluded in the field of view.

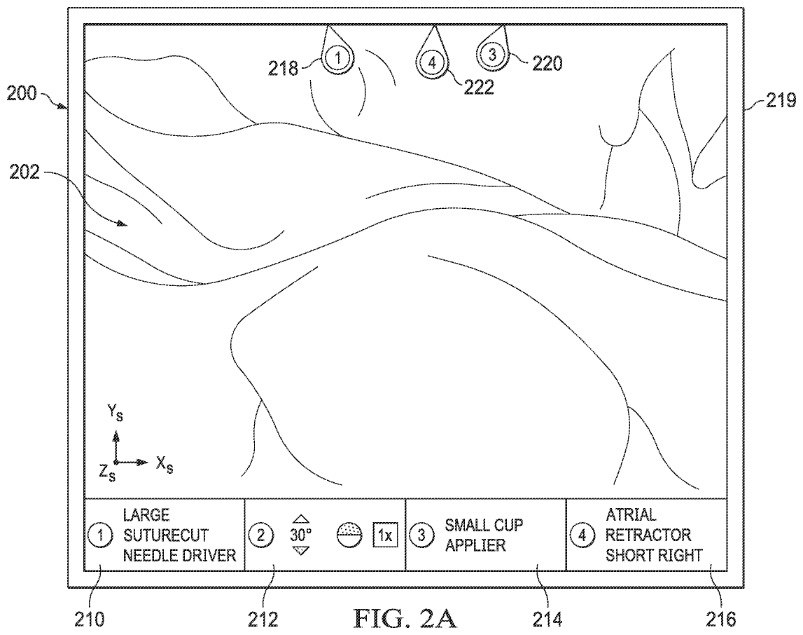

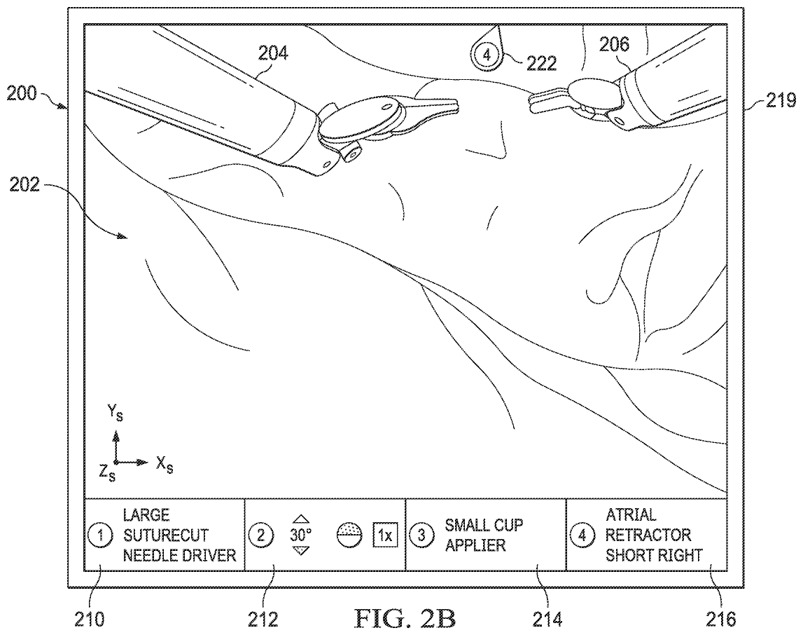

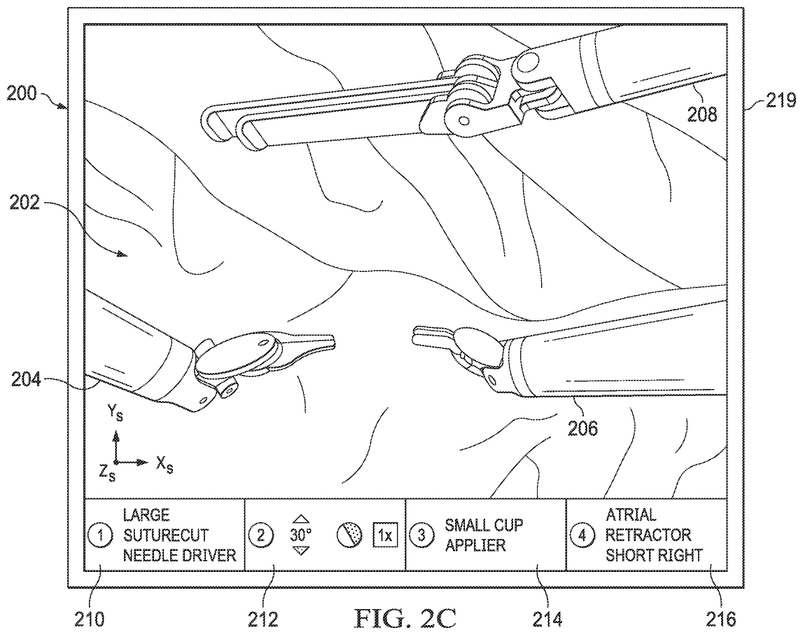

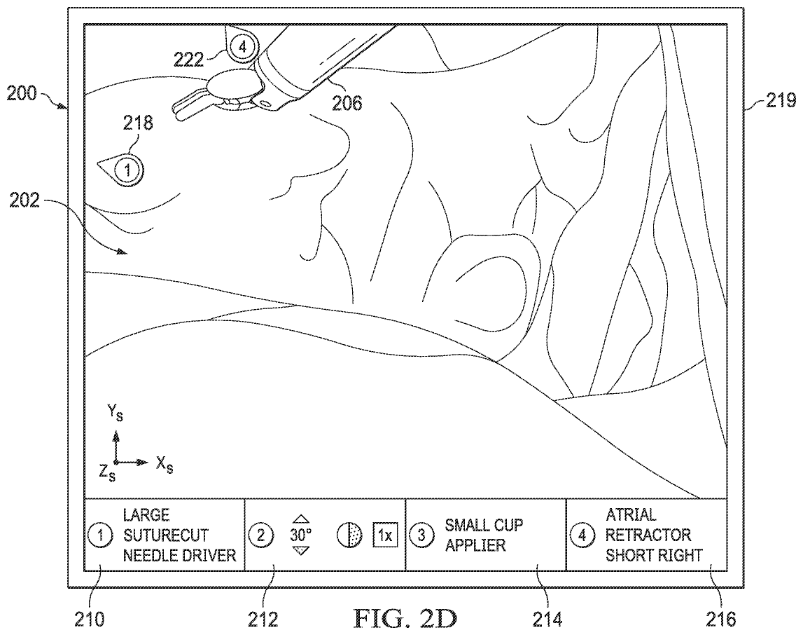

FIGS. 2A, 2B, 2C, and 2D illustrate a graphical user interface 200 that may be displayed, for example, on display system 35. The graphical user interface 200 may include a field of view portion 202 for displaying an image of a field of view 203 of a surgical environment 201 captured by an imaging system (e.g. imaging system 15). The surgical environment may have a Cartesian coordinate system X_{S}, Y_{S}, Z_{S}. The image in the field of view portion 202 may be a three-dimensional, stereoscopic image and may include patient tissue and surgical components including instruments such as a medical tool 204, a medical tool 206, and a medical tool 208. The graphical user interface 200 may also include an information block 210 displaying information about medical tool 204, an information block 212 displaying information about the imaging system capturing the image in the field of view portion 202, an information block 214 displaying information about the medical tool 206, and an information block 216 displaying information about the medical tool 208. The information blocks 210, 212, 214, 216 may include the tool type, the number of the manipulator arm to which the tool is coupled, status information for the arm or the tool, and/or operational information for the arm or the tool.

The graphical user interface 200 may also include one or more synthetic indicators 218, 220, 222 that may appear in the field of view portion 202 when a corresponding medical tool is in the surgical environment but outside the view of the imaging system and thus not visible in the field of view portion 202. The synthetic indicator 218 indicates the tool 204. The synthetic indicator 220 indicates the tool 206. The synthetic indicator 222 indicates the tool 208. Each synthetic indicator 218, 220, 222 may have a three-dimensional shape and may point in the three-dimensional direction of the corresponding tool outside of the field of view.

In FIG. 2A, the field of view portion 202 includes a three-dimensional image of a portion of a surgical environment, and synthetic indicators 218, 220, 222 at an image perimeter 219 point to respective tools 204, 206, 208 in the surgical environment but outside the field of view of the imaging system. In FIG. 2B, the imaging system (e.g. endoscope) has been moved in the +Y direction to capture a different image of the surgical environment in the field of view portion 202. The distal ends of tools 204 and 206 are now visible. The tool 208 remains outside the field of view and, consequently, the synthetic indicator 222 is displayed indicating the direction of the tool 208. In FIG. 2C, the imaging system has been moved further in the +Y direction to capture a different image of the surgical environment in the field of view portion 202. The distal ends of tools 204, 206, and 208 are now visible in the field of view portion 202. Thus, no synthetic indicators are displayed. In FIG. 2D, the imaging system has been moved in the −Y, +X directions to capture a different image of the surgical environment in the field of view portion 202. Tool 206 remains visible in the field of view portion 202 but the tools 204, 208 are now outside of the field of view portion 202. Thus, the synthetic indicators 218, 222 are displayed and point to the three-dimensional locations of the tools 204, 208, respectively, in the surgical environment.

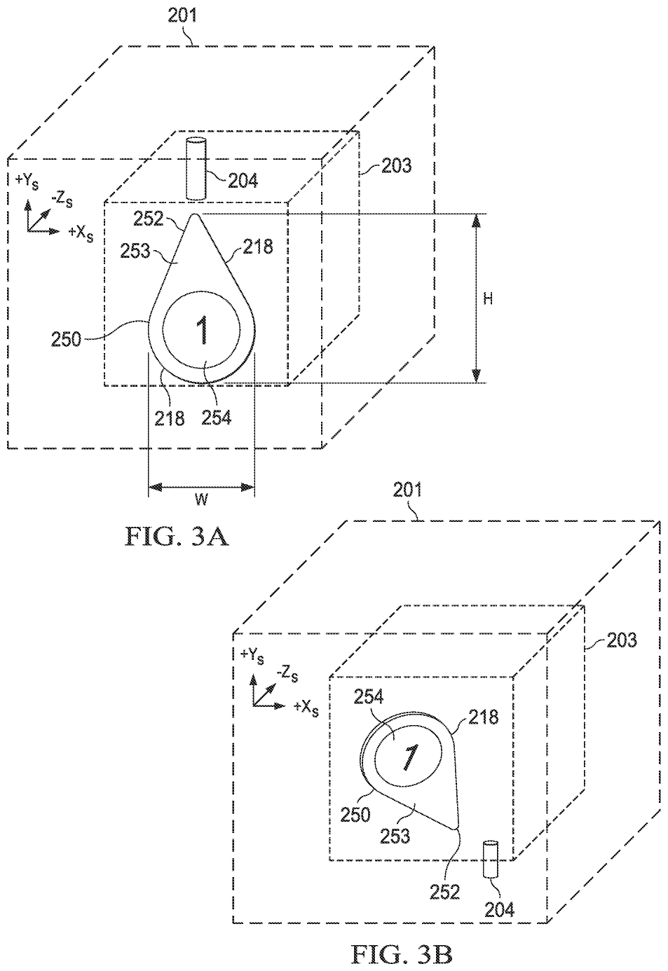

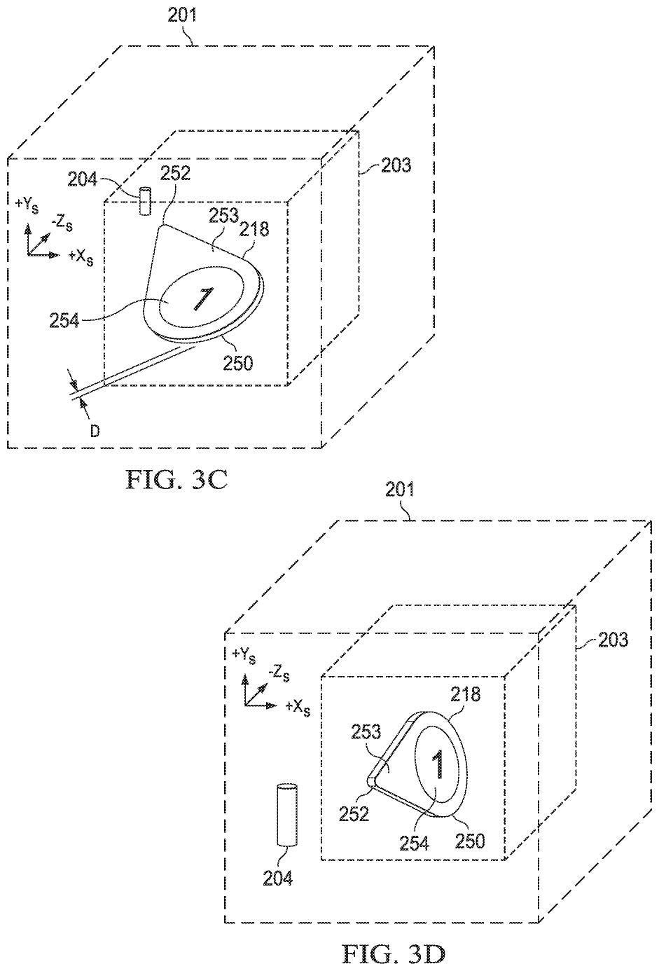

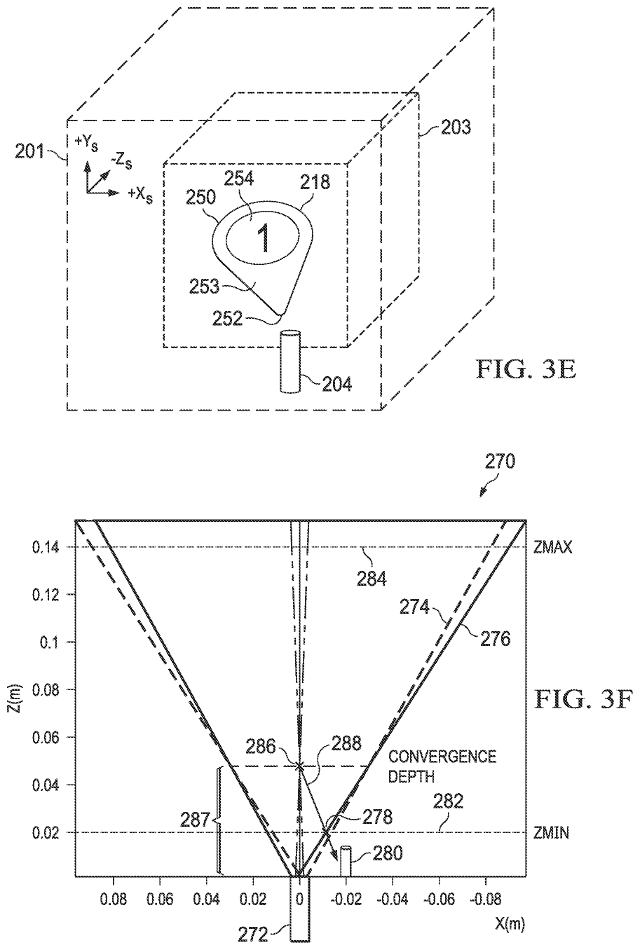

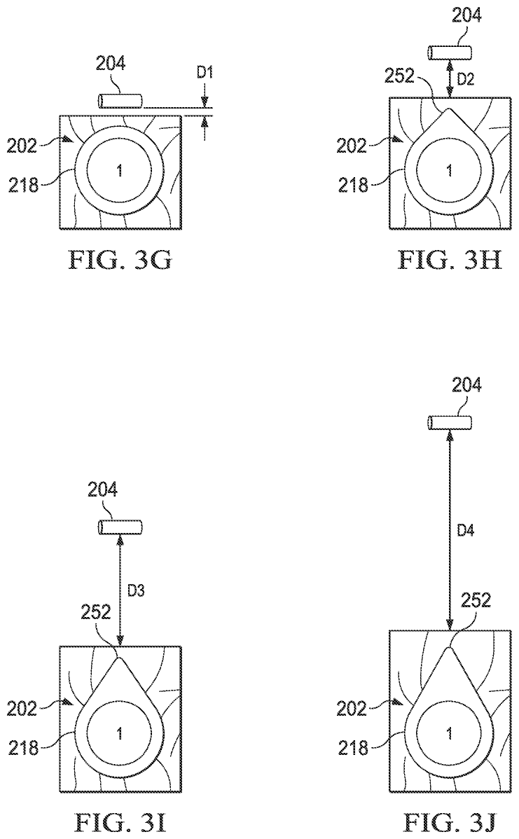

FIGS. 3A-3E illustrate the field of view 203 of the surgical environment 201 with the synthetic indicator 218 in various three-dimensional orientations to point to different locations of the medical tool 204. In each illustration, the medical tool 204 is in the surgical environment 201 but outside of the field of view 203 and thus the synthetic indicator 218 is displayed in the field of view portion 202. The synthetic indicator 218 includes an indicator body 250 including a directional portion 252. The directional portion 252 may include a taper that may point toward the medical tool 204. In this embodiment the synthetic indicator 218 may have a teardrop shape, but in other embodiments, arrows, triangles, or other pointed symbols capable of indicating direction may be used for the synthetic indicator. The indicator body 250 may have a three-dimensional shape with a height H, depth D, and width W dimensions. The indicator body 250 may have at least one flat surface 253 and an icon 254 that may appear as a decal affixed along the flat surface 253. The icon 254 may include an identifier such as an identification for the manipulator arm to which the indicated tool is coupled or an identification for the indicated tool itself. As the indicator body 250 moves in three-dimensional space, the orientation of the icon 254 may rotate relative to the indicator body and directional portion 252 so that text or symbols on the icon 254 remains upright to the viewer. The orientation of the icon 254 may also remain aligned with the orientation of the face of the indicator body 250.

As the tool 204 moves within the surgical environment 201 or as the field of view 203 changes within the surgical environment, the synthetic indicator 218 may pivot such that the directional portion 252 remains pointed toward the tool 204 and the flat surface 253 remains visible to the viewer. In FIG. 3A, the directional portion 252 is pointed toward the tool 204 located outside the field of view 203 in the +Y direction relative to the synthetic indicator 218. In FIG. 3B, the directional portion 252 is pointed toward the tool 204 located outside the field of view 203 in the −Y, +X, −Z direction relative to the synthetic indicator 218. In FIG. 3C, the directional portion 252 is pointed toward the tool 204 located outside the field of view 203 in the +Y, −X, −Z direction relative to the synthetic indicator 218. In FIG. 3D, the directional portion 252 is pointed toward the tool 204 located outside the field of view 203 in the −X, +Z direction relative to the synthetic indicator 218. In FIG. 3E, the directional portion 252 is pointed toward the tool 204 located outside the field of view 203 in the −Y, +X, +Z direction relative to the synthetic indicator 218. The examples of FIGS. 3D and 3E may depict the use of the synthetic indicator when a tool tip is behind the endoscope tip. In the absence of the pointing direction, the user might be confused about which direction to move the endoscope. For example, when a tool tip is just behind the endoscope tip, a lateral move of the endoscope by itself can be counter-productive and result in the projected tool indicator rapidly swinging from one side of the field of view to the other, making is difficult to locate the tool.

In some embodiments, the synthetic indicator 218 or a portion thereof may have a color coding or other visual treatment to indicate a status and/or a control mode (e.g., active or inactive; location where clutch initiated) of the associated tool 204. In some embodiments, the orientation of the synthetic indicator 218 may be determined based on presentation objectives including visibility to the viewer. For example, the flat surface 253 may be oriented toward the endoscope, and the icon 254 may be oriented in the place of the surface 253 to be upright in the view. The directional portion 252 may be constrained so that a normal to the flat surface 253 is oriented within a viewing cone or frustum of the endoscope to ensure legibility of the icon 254. The stereoscopic depth of the synthetic indicator 218 position may be constrained for ease of fusion, to reduce depth mismatch with endoscopic scene content, and to resolve occlusion and depth relative to other synthetic elements in the field of view portion 202. The apparent size of the synthetic indicator 218 may be constrained based on its depth.

In some embodiments, the position of the directional portion 252 along the perimeter 219 of the field of view portion 202 is computed by a ray intersection with the stereoscopic images. FIG. 3F provides a top-view of the stereoscopic viewing frustum 270 of an endoscope 272 (e.g. imaging system 15) providing the field of view portion 202. The stereoscopic viewing frustum 270 is formed from right eye frustum 274 which corresponds to the right eye field of view and from left eye frustum 276 which corresponds to the left eye field of view. A stereo convergence location 286 is at a convergence depth 287 from the distal tip of the endoscope. A marker 278 corresponds to a projected location for a tip of a directional portion of a three-dimensional synthetic indicator (e.g. indicator 218) that is pointing towards a keypoint on an instrument tip portion 280. The marker 278 location is resolved to be within a minimum depth range 282 and maximum depth range 284 of the distal tip of endoscope 272 and within the field of view frustums 274, 276 of both the left and right eyes. The minimum and maximum depth range determination may provide for stereoscopic viewing comfort as well as accentuate user perception of the relative spatial relationship of an offscreen tool. In this example with the instrument tip portion 280 located as shown, a tip of the directional portion of the synthetic indicator may appear at the marker 278, namely the intersection location of the minimum depth range 282 and the left eye frustum 276. The directional portion of the synthetic marker may be nominally aligned along a pointing direction 288 between the convergence location 286 and marker 278.

For example, a ray extending from a point along a centerline of an imaging component (e.g. an endoscope) to a distal keypoint on the associated instrument (e.g. a predetermined point on the instrument end effector or joint) may be determined. This determination resolves a point along the perimeter 219 that may be visible by both eyes within a comfortable depth range for fusion.

In some embodiments, the synthetic indicator may morph in shape and size as the endoscope and/or medical tools are moved. For example, the synthetic indicator may transition from a circular badge to the teardrop shape. The length of the teardrop or arrow shape may indicate the distance of the tool from the field of view. The synthetic indicator may also emphasize a direction and/or distance of travel to locate the offscreen tool. For example, when tools are located farther than a threshold distance from the field of view, all or a portion of the synthetic indicator may be animated to produce a gestural cue to emphasize that the tool is greater than a threshold distance from the field of view. FIGS. 3G-3J provide a progression of images depicting a modulation of the length of the synthetic indicator 218 in correspondence with the distance of the instrument tip 204 outside of the field of view portion 202 and importance of the direction of travel to the instrument tip 204. As shown in FIG. 3J, when the instrument tip 204 is at a distance D4 far outside of the field of view volume (e.g. greater than 3 cm) or its Z-direction position is outside of the minimum and maximum depth ranges, then the synthetic indicator, and in particular the directional portion 252, is lengthened or otherwise accentuated to convey both direction and distance of travel. By contrast and as shown in FIG. 3G, if the instrument tip 204 is close to the field of view (e.g. <3 cm) and its Z position is within the minimum and maximum depth ranges, then the directional portion 252 is de-emphasized such that the synthetic indicator 218 becomes more circular. In the example of FIG. 3G, the position along the perimeter of the display is sufficient to depict the lateral spatial location of the instrument tip 204 with respect to the endoscope view volume. FIG. 3H illustrates a directional portion 252 that is more pronounced and longer than in FIG. 3G, indicating that the distance D2 to the instrument tip 204 is greater than the distance D1. FIG. 3I illustrates a directional portion 252 that is longer than in FIG. 3H, but not as long as in FIG. 3J, indicating that the distance D3 to the instrument tip 204 is greater than distance D2 but not as long as distance D4.

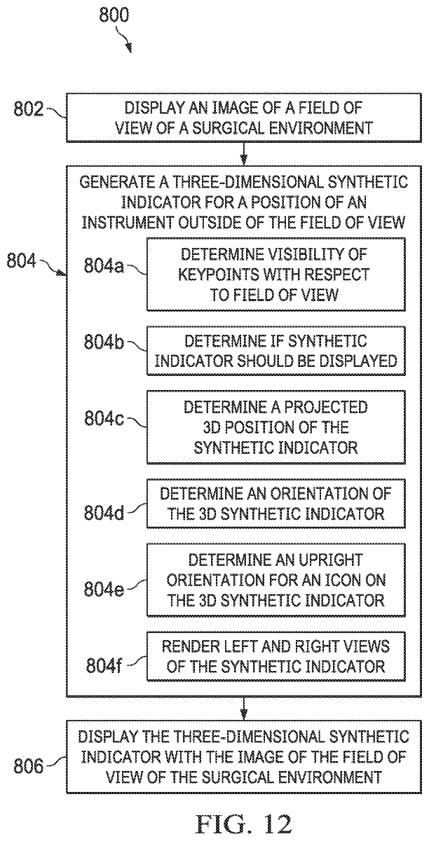

A method 800 for displaying a three-dimensional synthetic indicator (e.g., a synthetic indicator 218, 220 or 222) is illustrated in the flowchart of FIG. 12. The methods described herein are illustrated as a set of operations or processes and are described with continuing reference to the additional figures. Not all of the illustrated processes may be performed in all embodiments of the methods. Additionally, one or more processes that are not expressly illustrated in may be included before, after, in between, or as part of the illustrated processes. In some embodiments, one or more of the processes may be implemented, at least in part, in the form of executable code stored on non-transitory, tangible, machine-readable media that when run by one or more processors (e.g., the processors of a control system) may cause the one or more processors to perform one or more of the processes. In one or more embodiments, the processes may be performed by a control system.

At a process 802, an image of the field of view (e.g., field of view portion 202) in a surgical environment (e.g. surgical environment 201) is displayed on, for example a display 35. In some embodiments, the process 802 may include one or more of the process 804a-804f. At a process 804a, the visibility of the instrument tip keypoints with respect to an endoscope field of view volume may be determined.

At a process 804b, a determination may be made about whether a synthetic indicator should be displayed for an offscreen instrument based on context and predetermined rules. Displaying the synthetic indicator at all times while a tool tip is outside of the field of view may introduce undesirable distractions. Therefore, predetermined rules may be imposed on when the synthetic indicator is shown so that it is more contextual and its visibility coincides with operator workflow steps that benefit from user awareness of the offscreen tool location. For example, the synthetic indicator may be displayed when endoscope movement is active either from bedside or the surgeon console. The synthetic indicator may be displayed when a guided tool change feature is active on the tool's manipulator arm. The synthetic indicator may be displayed when an instrument clutch is active for the manipulator arm controlling an offscreen tool. The synthetic indicator may be displayed when a surgeon console user is about to start control of a manipulator arm that controls an offscreen tool. The synthetic indicator may be displayed when the surgeon console user has started control of a manipulator arm that controls an offscreen tool. The synthetic indicator may be displayed when the surgeon console user is changing hand association to a manipulator arm coupled to an offscreen tool. The synthetic indicator may be displayed when a notification is displayed for a manipulator arm to which an offscreen tool is coupled.

At a process 804c, a projected three-dimensional position of the synthetic indicator along lateral extents of the field of view volume may be determined. At a process 804d, an orientation of the three-dimensional synthetic indicator may be determined to face the endoscope tip within the visibility cone or frustum. At a process 804e, an upright orientation of the icon (e.g. icon 254) on surface of the synthetic indicator may be computed. At a process 804f, both left and right views of the synthetic indicator may be rendered using a calibrated stereoscopic camera model that corresponds to the endoscope optics.

At a process 804, a three-dimensional synthetic indicator (e.g., indicator 218) indicating a position of an instrument outside of the field of view may be generated. More specifically, in some embodiments, a composite rendering of the left and right synthetic indicators may be overlayed on the endoscopic video.

At a process 806, the three-dimensional synthetic indicator may be displayed with the image of the field of view of the surgical environment.

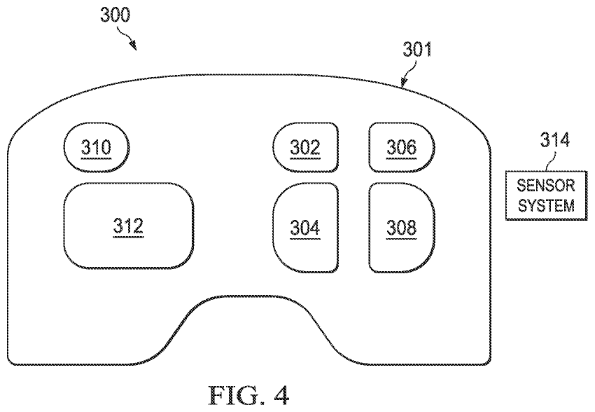

FIG. 4 provides a top view of an input control apparatus 300 of an operator input system (e.g. operator input system 16) that includes an input panel 301 which forms a common platform for input control devices 302, 304, 306, 308, 310, 312 (e.g. input control devices 37) which are configured as foot pedals that receive input from a user's foot. The foot pedals 302, 304, 306, 308, 310, 312 may be engaged to control functions of a teleoperational assembly (e.g. assembly 12) and/or medical tools coupled to the arms of the teleoperational assembly. The input control apparatus 300 may also include a sensor system 314 that detects a position of a user (e.g., the user's foot or leg) relative to the input control devices. The sensor system 314 may include cameras, optical sensors, motion sensors or other sensors that sense or track user presence at or near one or more of the input control devices 302-312. The sensor system 314 may also include pressure sensors, displacement sensors, or other types of sensors that detect that one or more of the input control devices has been activated or engaged.

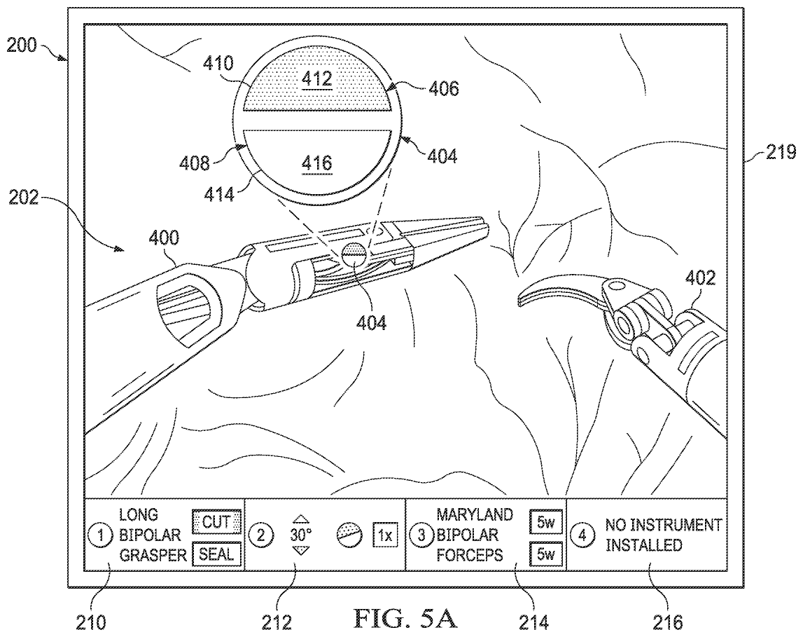

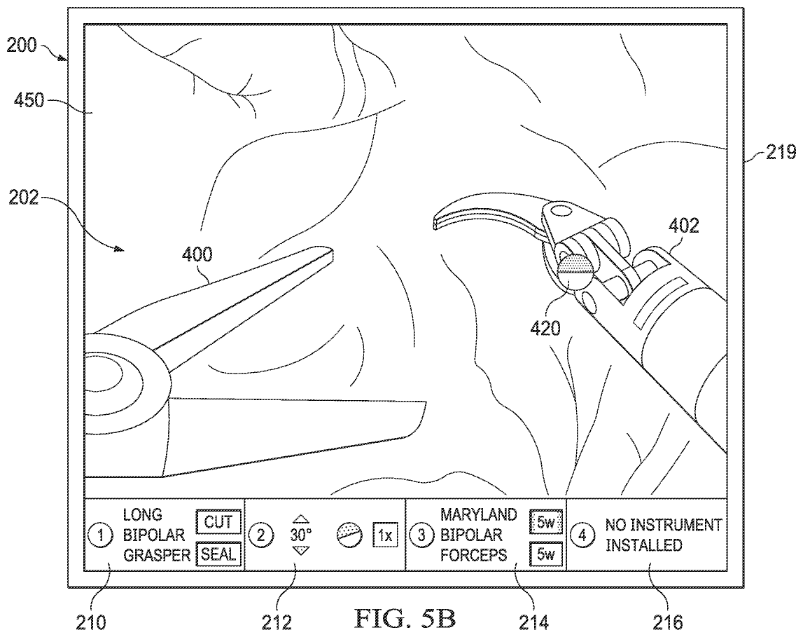

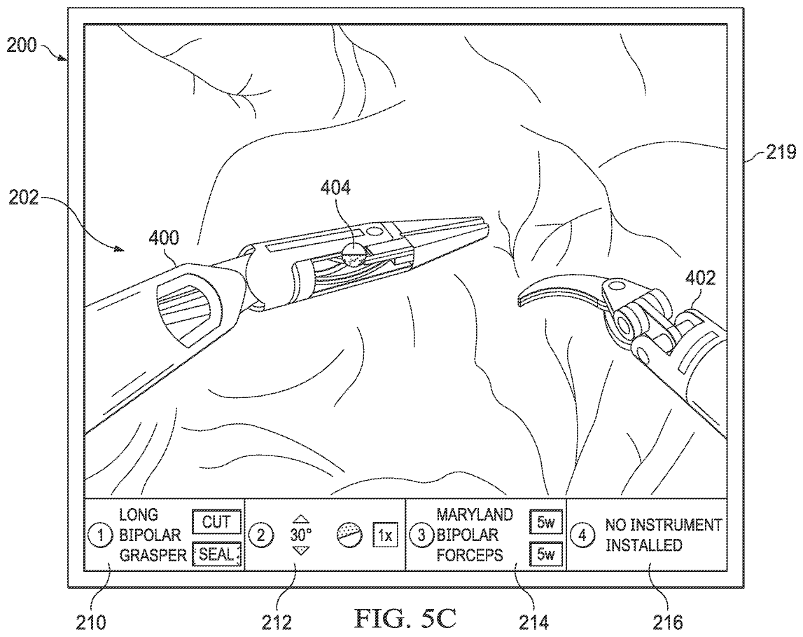

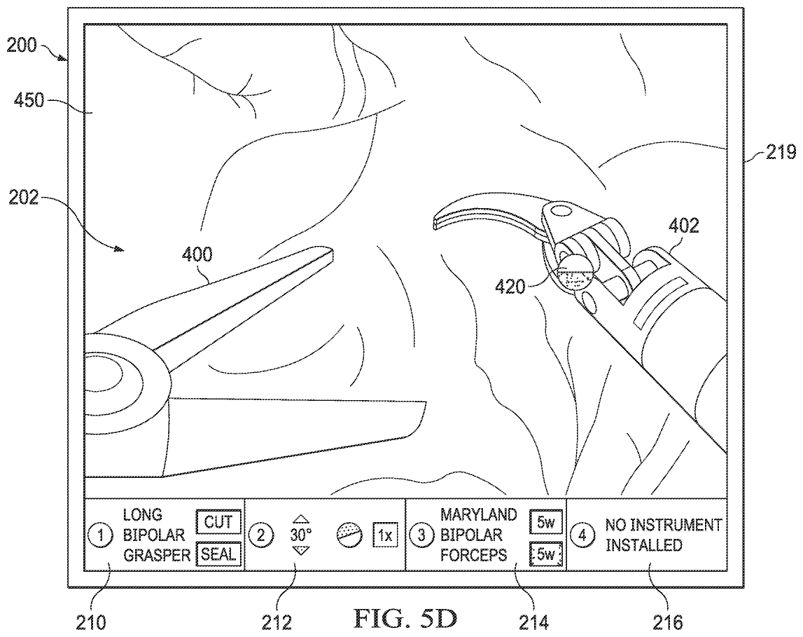

FIGS. 5A, 5B, 5C, and 5D illustrate the graphical user interface 200. A medical tool 400 and a medical tool 402 are visible in the field of view portion 202. Functions of the medical tools may be initiated by engaging corresponding foot pedals on the input panel 301. For example, the medical tool 400 may be operated by manipulator arm 1 as indicated in information block 210 and may be a vessel sealer that may perform the function of cutting when the foot pedal 302 is engaged and may perform the function of sealing when the foot pedal 304 is engaged. As shown in FIG. 5A, the tool 400 may be labeled with a synthetic indicator 404. In this embodiment, the synthetic indicator 404 may be a generally circular badge including an upper semi-circular portion 406 and a lower semi-circular portion 408. The upper semi-circular portion 406 includes an outline portion 410 and a central portion 412, and the lower semi-circular portion 408 includes an outline portion 414 and a central portion 416. The upper semi-circular portion 406 may correspond to the function of the secondary foot pedal 302 and may indicate the engagement status (e.g., hovered, activated) of the pedal 302. The lower semi-circular portion 408 may correspond to the function of the primary foot pedal 304 and may indicate the engagement status (e.g., hovered, activated) of the pedal 304. The spatial relationship of the upper semi-circular portion 406 and the lower semi-circular portion 408 may have the same or a similar spatial relationship as the pedals 302, 304. When the sensor system 314 detects that an operator's foot is hovering above or otherwise within a threshold distance from the foot pedal 302, the outline portion 410 of the upper semi-circular portion 406 may change appearance (e.g., change color, become animated) to indicate to the operator that the operator's foot is near the foot pedal 302. Thus, the operator can determine the foot position while the operator's vision remains directed to the graphical user interface 200. When the operator engages the foot pedal 302 (e.g., steps on or depresses the pedal), the central portion 412 of the upper semi-circular portion 406 may change appearance (e.g., change color, become animated) to indicate to the operator that the operator's foot has engaged the foot pedal 302 and the function of the foot pedal 302 (e.g., cutting) has been initiated. In some embodiments, the hover or engaged status of the foot pedal 302 may be indicated in the information block 210 using the same or similar graphical indicators. The left bank of foot pedals (e.g., pedals 302, 304) may be associated with left hand input control devices, and the right bank of foot pedals (e.g., pedals 306, 308) may be associated with right hand input control devices. Each hand may be associated to control any instrument arm. The co-located synthetic indicators reflect this association of an instrument to a corresponding hand & foot. In some configurations, the instrument pose with respect to the endoscopic field of view may otherwise appear to have an ambiguous association to a left or right side, so the co-located synthetic indicator clarifies this association.

As shown in FIG. 5C, the lower semi-circular portion 408 may function, similarly to the upper semi-circular portion 406, as an indicator for the hover and engagement of the foot pedal 304. When the operator engages the primary foot pedal 304 (e.g., steps on or depresses the pedal), the central portion of the lower semi-circular portion 408 may change appearance (e.g., change color, become animated) to indicate to the operator that the operator's foot has engaged the foot pedal 304 and the function of the foot pedal 304 (e.g., sealing) has been initiated. The pedals at the surgeon's console may be color-coded. For example, primary pedals 304, 308 may be colored blue and the secondary pedals 302, 306 may be colored yellow. This color-coding is reflected in the associated highlight and fill colors of the pedal function synthetic indicators on the graphical user interface.

As shown in FIG. 5B, the tool 402 may be labeled with a synthetic indicator 420. In this embodiment, the synthetic indicator 420 may be substantially similar in appearance and function to the synthetic indicator 404 but may provide information about the set of foot pedals 306, 308. The tool 402 may be operated by manipulator arm 3 as indicated in information block 214 and may be a monopolar cautery instrument that may perform the function of delivering an energy for cutting when the foot pedal 306 is engaged and may perform the function of delivering an energy for coagulation when the foot pedal 308 is engaged. When the sensor system 314 detects that an operator's foot is hovering above or otherwise within a threshold distance from the secondary foot pedal 306, an outline portion of an upper semi-circular portion may change appearance to indicate to the operator that the operator's foot is near the foot pedal 306. When the sensor system 314 determines that the operator has engaged or activated the foot pedal 306, a central portion of the upper semi-circular portion may change appearance to indicate to the operator that the operator's foot has engaged the foot pedal 306 and the function of the foot pedal 306 (e.g., delivering energy for cutting) has been initiated. In some embodiments, the hover or engaged status of the secondary foot pedal 302 may be indicated in the information block 214 using the same or similar graphical indicators.

As shown in FIG. 5D, the lower semi-circular portion of indicator 420 may function, similarly to the upper semi-circular portion, as an indicator for the hover and engagement of the primary foot pedal 308. When the operator engages the primary foot pedal 308, the central portion of the lower semi-circular portion may change appearance to indicate to the operator that the operator's foot has engaged the primary foot pedal 308 and the function of the foot pedal 308 (e.g., delivering energy for coagulation) has been initiated.

The position and orientation of synthetic indicators 404, 420 may be determined to create the appearance that the synthetic indicators are decals adhered, for example, to the tool clevis or shaft. As the tools or endoscope providing the field of view are moved, the synthetic indicators 404, 420 may change orientation in three-dimensional space to maintain tangency to the tool surface and to preserve the spatial understanding of upper and lower pedals.

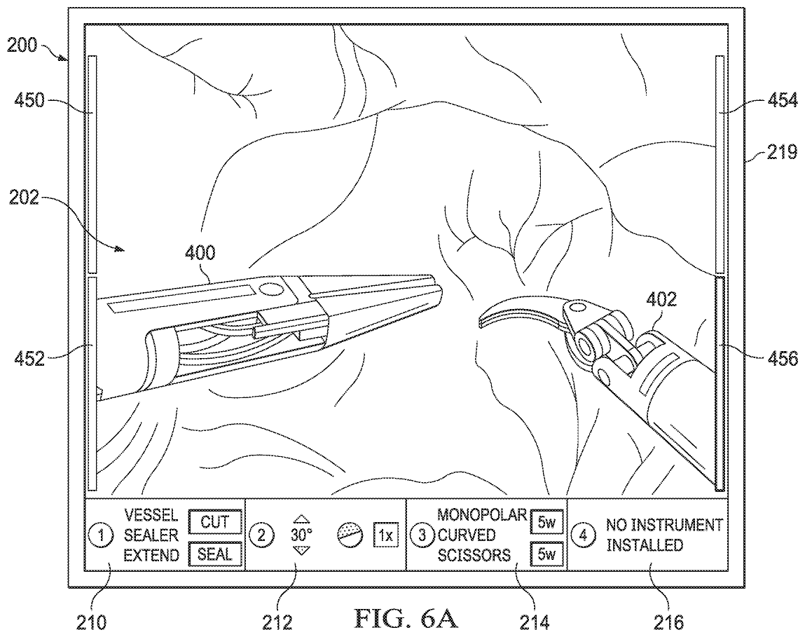

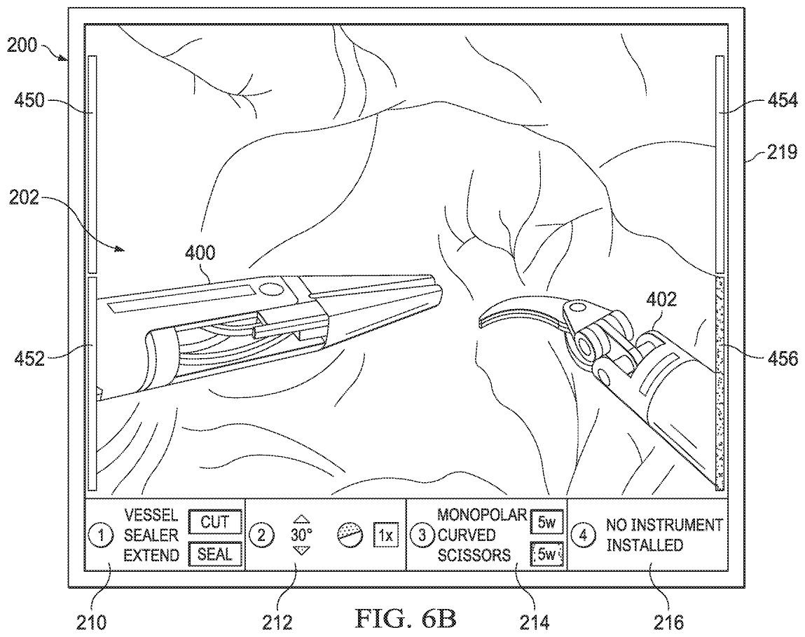

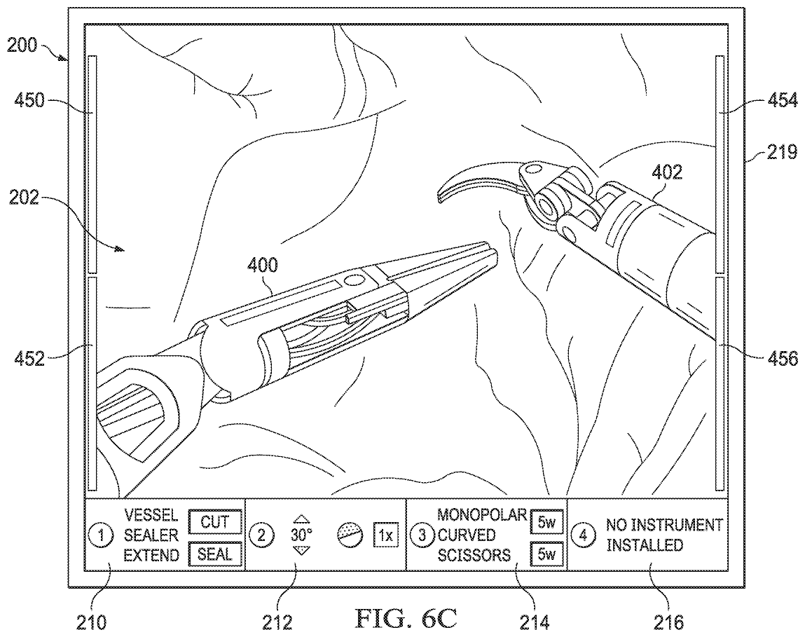

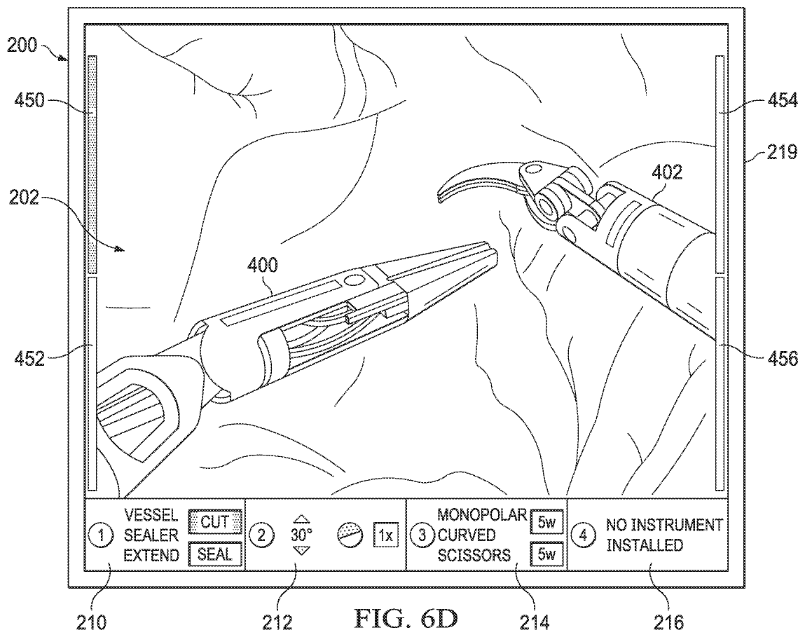

Various types, shapes, and configurations of synthetic indicators may be displayed to provide information about the status of foot pedal engagement. In an alternative embodiment, as shown in FIGS. 6A, 6B, 6C, and 6D, the graphical user interface 200 with medical tools 400, 402 is visible in the field of view portion 202. In this embodiment, synthetic indicators 450, 452, 454, 456 may take the form of elongated bars that extend along the perimeter 219. In this example, the synthetic indicators 450-456 are inside the boundary of the perimeter 219, but in alternative embodiments may be outside the perimeter 219 of the field of view 202.

In this embodiment, the synthetic indicator 450, 452 may perform a function similar to synthetic indicator 404 in providing information about the set of foot pedals 302, 304. As shown in FIG. 6A, when the sensor system 314 detects that an operator's foot is hovering above or otherwise within a threshold distance from the primary foot pedal 308, the synthetic indicator 456 is outlined, indicating to the operator that the operator's foot is near the primary foot pedal 308. As shown in FIG. 6B, when the operator engages the foot pedal 308, the synthetic indicator 456 may become a filled bar to indicate to the operator that the operator's foot has engaged the foot pedal 308 and the function of the foot pedal 308 has been initiated. In some embodiments, the hover or engaged status of the foot pedal 308 may be indicated in the information block 214 using the same or similar graphical indicators.

As shown in FIG. 6C, when the sensor system 314 detects that an operator's foot is hovering above or otherwise within a threshold distance from the secondary foot pedal 302, the synthetic indicator 450 is outlined, indicating to the operator that the operator's foot is near the foot pedal 302. As shown in FIG. 6D, when the operator engages the foot pedal 302, the synthetic indicator 456 may become a filled bar to indicate to the operator that the operator's foot has engaged the foot pedal 302 and the function of the foot pedal 302 has been initiated. In some embodiments, the hover or engaged status of the foot pedal 302 may be indicated in the information block 210 using the same or similar graphical indicators.

In alternative embodiments, audio cues may be provided instead of or in addition to the synthetic indicators to provide instructions or indicate spatial direction (e.g., up/down/left/right) to move the operator's foot into a hover position for a foot pedal. The system may distinguish between hovering a foot over a pedal vs. actuating the pedal, and there may be distinct visual and audio cues for hover status versus the engaged or actuation status. The system may also depicts when a pedal function is valid or invalid. The highlight color may appears in gray when a pedal function is not valid (e.g. when the instrument function cable not plugged in or the instrument function is not configured)

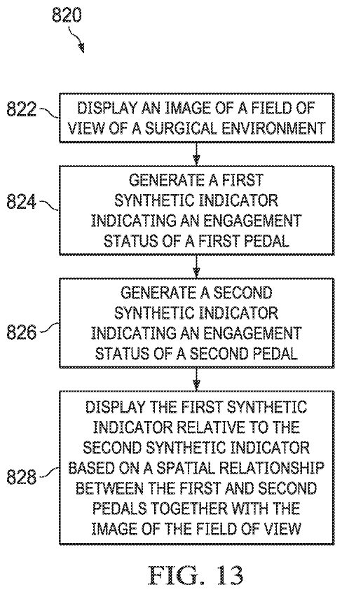

A method 820 for displaying synthetic indicators corresponding to a set of foot pedals is illustrated in the flowchart of FIG. 13. At a process 822, an image of a field of view (e.g., a field of view portion 202) of a surgical environment (e.g. environment 201) is displayed, for example on the display 35. At a process 824, a first synthetic indicator (e.g., the semi-circular portion 406) indicating an engagement status of a first pedal 302 is generated. At a process 826, a second synthetic indicator (e.g., the semi-circular portion 408) indicating an engagement status of a second pedal 304 is generated. At a process 828, the first synthetic indicator is displayed relative to the second synthetic indicator based on a spatial relationship between the first and second pedals. The first and second indicators are displayed with the image of the field of view.

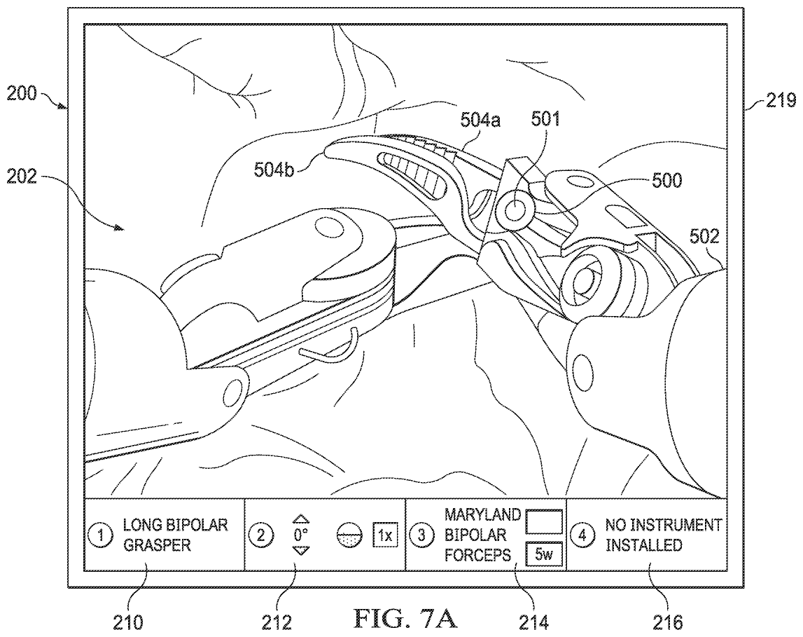

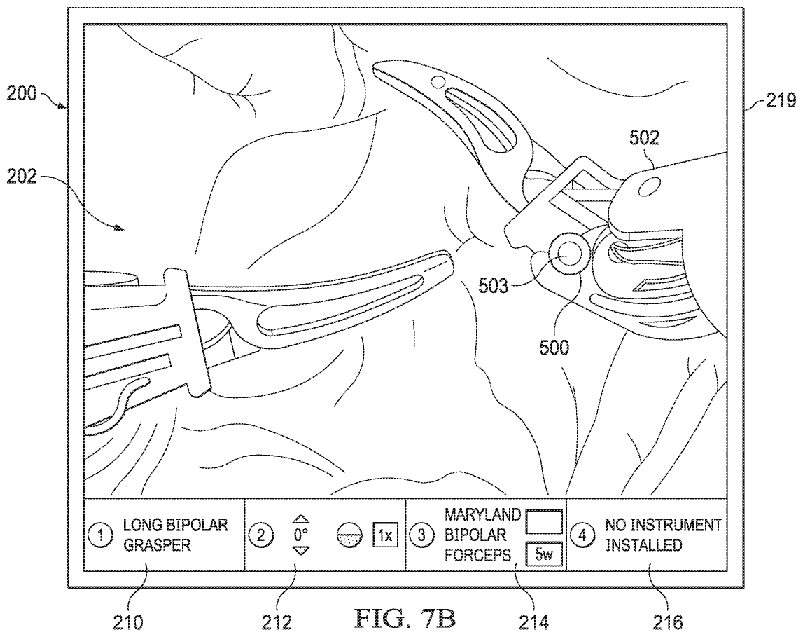

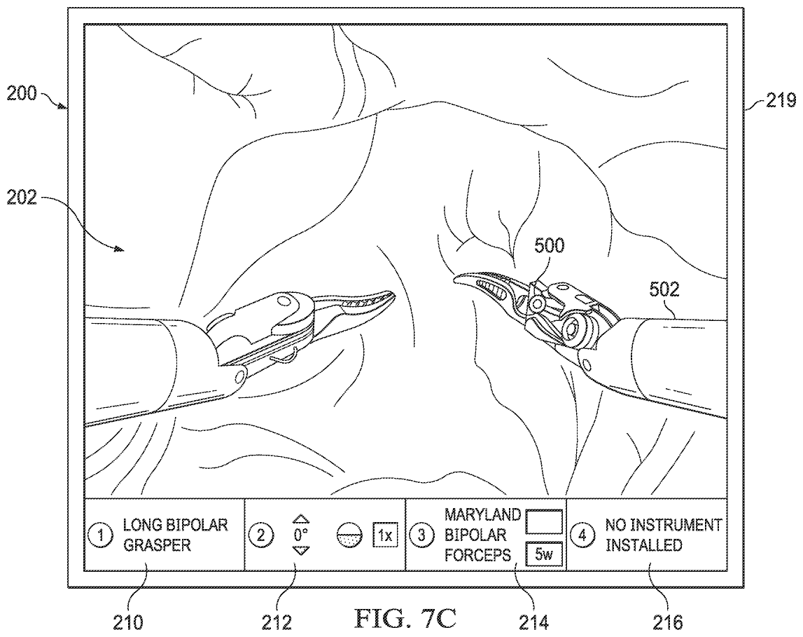

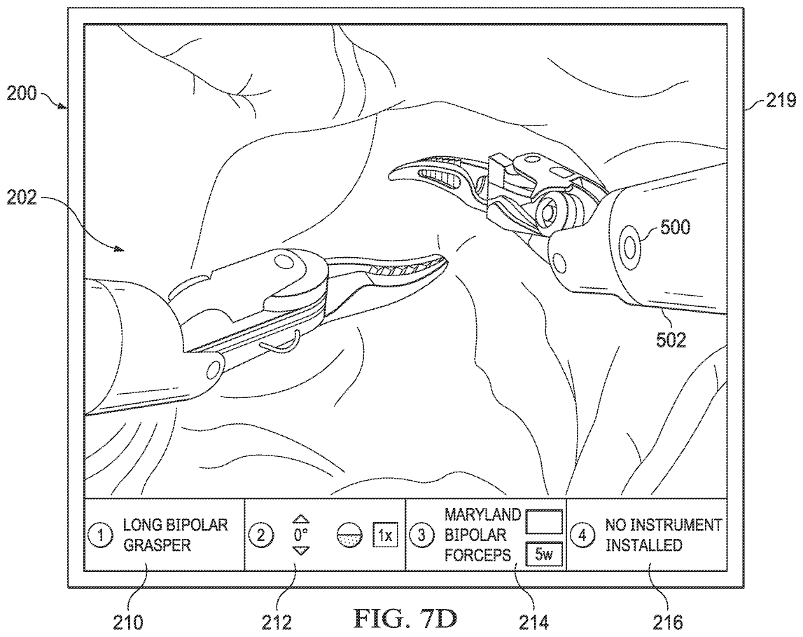

As shown in FIGS. 7A-7D, synthetic indicators that display as badges or labels on components in the field of view portion 202 may appear in proximity to the components and may conditionally move to stay visible and in proximity to the components as the components or the endoscope generating the field of view are moved. Synthetic indicators may be used for any of the purposes described above but may also be used to identify medical tools or other components in the field of view portion 202, identify the manipulator arm to which the medical tool is coupled, provide status information about the medical tool, provide operational information about the medical tool, or provide any other information about the tool or the manipulator arm to which it is coupled.

As shown in FIG. 7A, a synthetic indicator 500 may be associated with a tool 502. In this embodiment, the synthetic indicator 500 may be a badge configured to have the appearance of a decal on the tool 502. The badge 500 may appear in proximity to jaws 504a, 504b of the tool 502, but may be positioned to avoid occluding the jaws. The placement may include a bias away from the jaws based on the positional uncertainty of the underlying kinematic tracking technology. The default location of the badge 500 may be at a predetermined keypoint 501 on the tool 502. As shown in FIG. 7A, the badge 500 may be placed at a key point 501 located at a clevis of the tool. The badge 500 may pivot and translate as the endoscope or the tool 502 moves so that the badge 500 remains at the keypoint and oriented along a surface of the clevis. When the surface of the clevis is no longer visible in the field of view portion 202, the badge 500 may be moved to another keypoint 503 such as shown in FIG. 7B (at a predetermined joint location) or as shown in FIG. 7D (along the shaft of the tool 502).

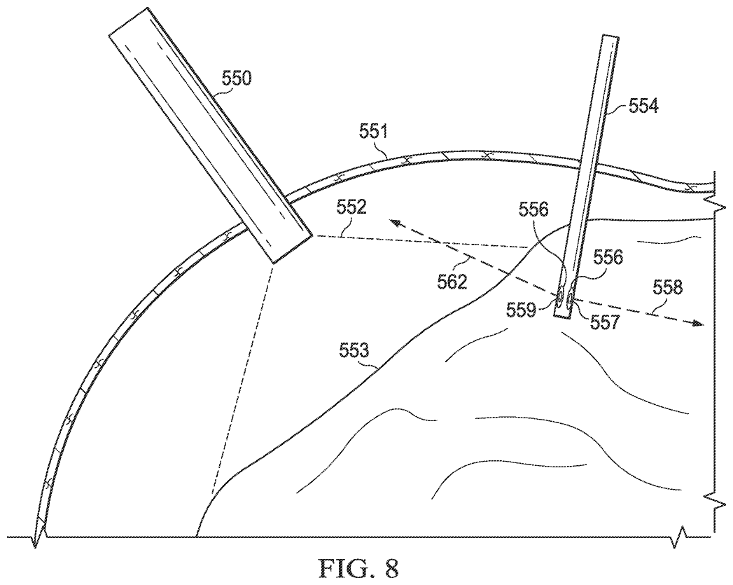

The badge 500 may remain at the original keypoint location if the keypoint location remains visible in the field of view portion 202. FIG. 8 illustrates an endoscope 550 (e.g., imaging system 15) extending into a patient anatomy 551. A viewing cone 552 extends from the distal end of the endoscope 550 to a tissue surface 553. The area in the viewing cone 552 may be the area visible in the field of view portion 202. A tool 554 extends into the patient anatomy 551. A badge 556 may have a default position at a keypoint 557. To determine if the default position is visible on the display, a line 558 normal to the surface of the badge 556 may be considered. Because the normal 558 does not extend within the viewing cone 552, a determination may be made that the badge 556 at the default position is not visible. Based on the condition that the normal 558 does not extend within the viewing cone 552, the badge 556 may be relocated to a secondary default position at a keypoint 559. A normal line 562 to the badge 556 at the second keypoint 559 is within the viewing cone 552 so the badge 556 may remain at the second keypoint 559 until movement of the tool 554 or the endoscope 550 causes a normal to the badge to no longer extend within the viewing cone 552. With reference again to FIG. 7B, because a normal to the badge 500 at the original keypoint (in FIG. 7A) is no longer within a viewing cone, the badge 500 may be relocated to a second default keypoint.

The orientation of the badge 500 at a keypoint may be constrained so that the normal to the badge surface is within the viewing cone and thus is visible in the field of view portion 202. If the badge 500 may not be oriented at a keypoint such that the normal is within the viewing cone, the badge 500 may be moved to a different keypoint. As shown in FIG. 7D, the orientation of the badge 500 may be pivoted to match the orientation of the tool 502 shaft while the surface of the badge 500 remains visible to the viewer. The size of the badge 500 may also change as the distance of the keypoint to which it affixed moves closer or further from the distal end of the endoscope or when a zoom function of the endoscope is activated. The badge size may be governed to stay within maximum and minimum thresholds to avoid becoming too large or too small on the display. As shown in FIG. 7C, the badge 500 may be smaller because the keypoint in FIG. 7C is further from the endoscope than it is in FIG. 7A.

The position, orientation, and depth of synthetic indicators associated with tools in the surgical environment may be determined based upon tool tracking by the control system and depth map analysis. Tool tracking alone may generate some residual error that may cause the synthetic indicators to appear to float over or interpenetrate the tool surface. This may be distracting to the viewer and may lead to fusion issues with the synthetic indicator and the associated tool. A depth map that provides information about the distance of the surfaces in the field of view portion 202 from the distal end of the endoscope may be used to refine placement of the synthetic indicator on the tool surface. More specifically, a raycast projection may be computed within a tolerance of a reference synthetic indicator position. The produced error may be used to estimate a radial offset correction for more accurately placing the synthetic indicator on the surface of the tool. The depth map quality and accuracy may be better when the tool is static or quasi-static, as compared to when the tool is moving. Thus, the raycasting and updating of the radial offset correction may be performed when the instrument keypoint velocity is lower than a threshold velocity. Alternatively, projective texturing may be used to place the synthetic indicator directly onto an extracted depth map surface. Systems and method for generating a depth map are further described in U.S. Pat. Nos. 7,907,166 and 8,830,224 which are incorporated by reference herein in their entirety.

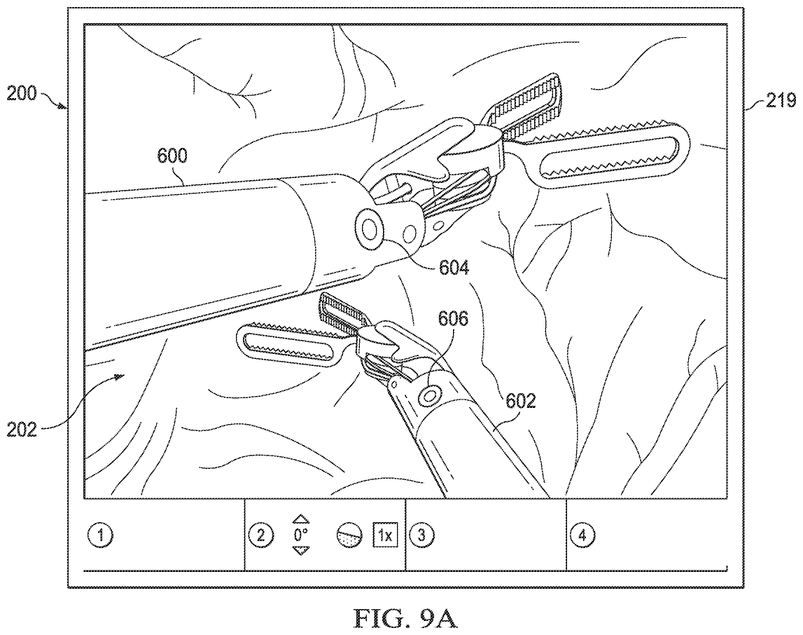

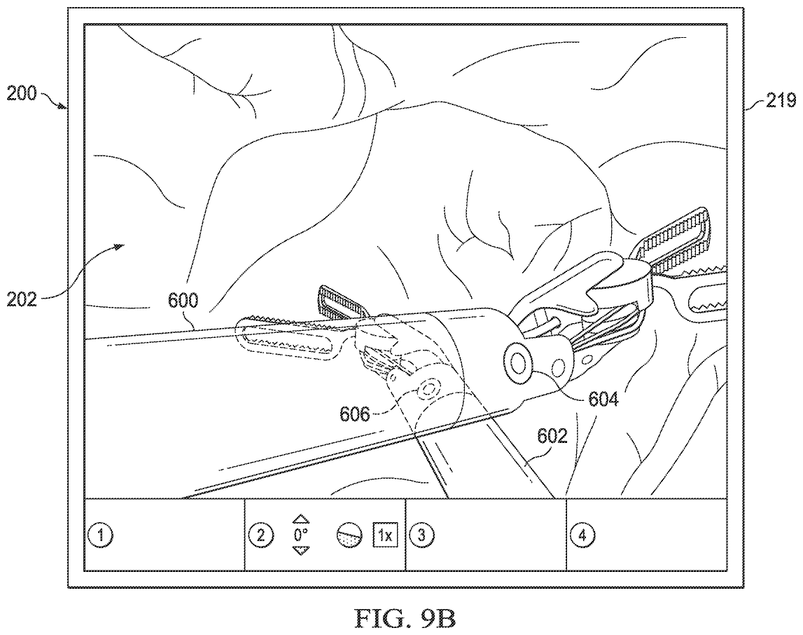

FIGS. 9A and 9B illustrate the graphical user interface 200 with a medical tool 600 and a medical tool 602 visible in the field of view portion 202. A synthetic indicator 604 is displayed on the medical tool 600, and a synthetic indicator 606 is displayed on the medical tool 602. As shown in FIG. 9B, as the tool 602 moves behind the tool 600 from the viewpoint of the endoscope, the position and orientation of the synthetic indicator 606 relative to the tool 602 may be maintained at the same three-dimensional depth as the surface of the tool 602 to which is appears fixed. The synthetic indicator 606 remains visually co-located with its keypoint even when positioned behind another object. Rather than being occluded by the tool 600, the synthetic indicator 606 may be shown with a visual treatment (e.g., ghosted appearance, faded appearance, translucent, dotted border) that indicates to the viewer that the synthetic indicator 606 is being viewed through a semi-opaque shaft of the tool 600. A depth map may be used to perform depth aware blending of the synthetic indicator 606 with the image of the field of view.

Three-dimensional synthetic indicators that are merely superimposed on a stereoscopic image, without depth consideration, may intersect or be occluded by content in the field of view. This can result in misleading spatial relationships between real and synthetic objects as well as stereoscopic fusion difficulties. Use of a depth map may improve the spatial appearance of synthetic indicators placed in the field of view. In some embodiments, depth mapping may be used for occlusion culling which causes portions of synthetic indicators that are deeper than the depth map to not be rendered and displayed. Complete or even partial culling of a synthetic indicator may result in a loss of physical co-location status information. In some examples, when a co-located synthetic indicator is being displayed in the presence of sub-optimal tracking or rendering conditions (e.g. depth occlusion, field of view culling, poor tracking performance, poor depth map quality, poor stereoscopic alignment, etc.), the graphical user interface may gradually fall back from the co-located indicators shown in FIGS. 5A-5D to the spatially-aligned peripheral indicators shown in FIGS. 6A-6D.

In other embodiments when using a depth map, the full synthetic indicator may be preserved, but otherwise occluded portions of the synthetic indicator may be rendered with a visual treatment (e.g., a translucent treatment) that differs from the unoccluded portions. To accomplish the special visual treatment for the occluded portion of a synthetic indicator, the rendering to the synthetic indicator may occur in two stages. In a first stage, the synthetic indicator may be rendered with a reduced opacity and without reference to or modification based on a depth map. In a second stage, the synthetic indicator may be rendered more opaquely while applying a depth map culling so that only pixels that are unoccluded appear more opaquely and are rendered over the pixels generated in the first stage. Thus, the occluded portions of the synthetic indicator appear with reduced opacity (e.g., more translucent) and the unoccluded portions of the synthetic indicator appear with greater or full opacity. In some embodiments, with use of a stereoscopic display, the synthetic indicator rendering for one eye (e.g., the viewer's non-dominant eye) may cull the occluded portions of the synthetic indicator, and the synthetic indicator rending for the other eye (e.g., the viewer's dominant eye) may be generated using a depth map and depth-aware blending. In some embodiments, a synthetic indicator may be generated based on a user generated graphic. The user generated graphic may be based in single eye image when the synthetic indicator is generated stereoscopically.

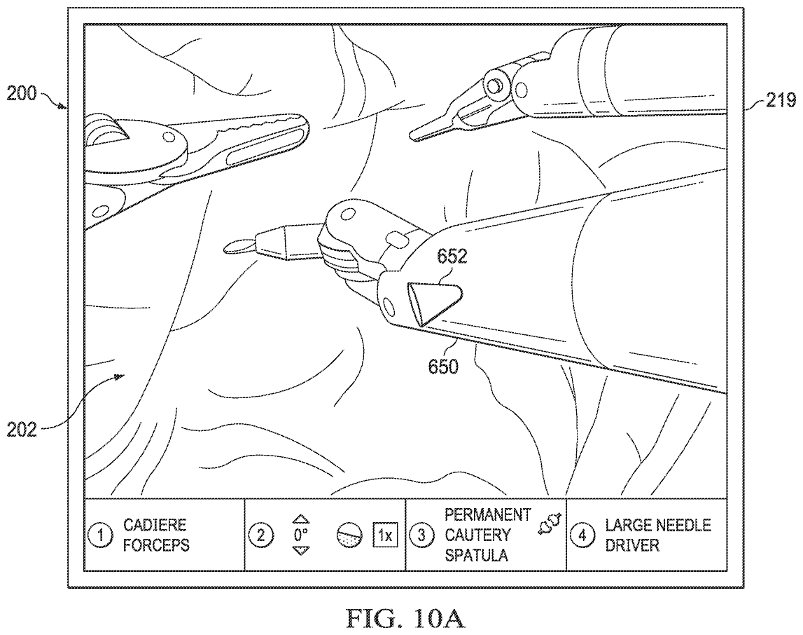

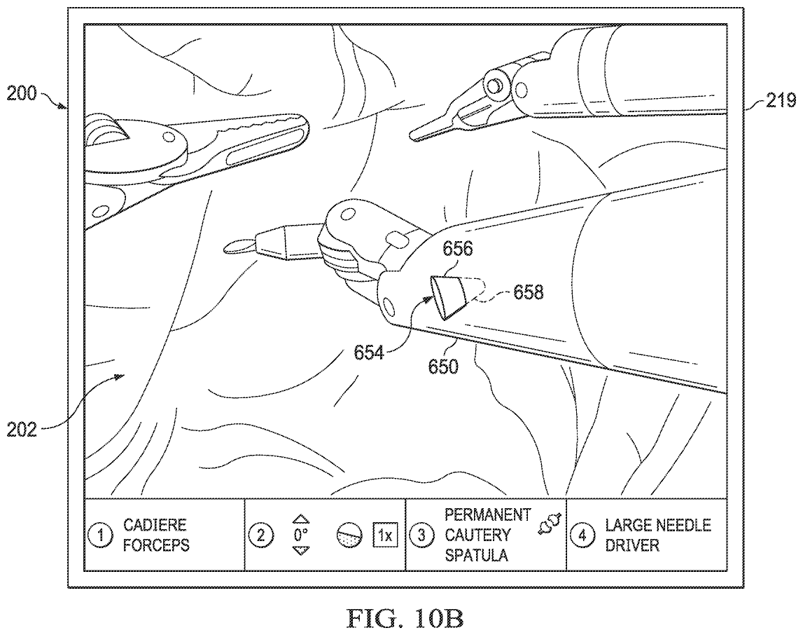

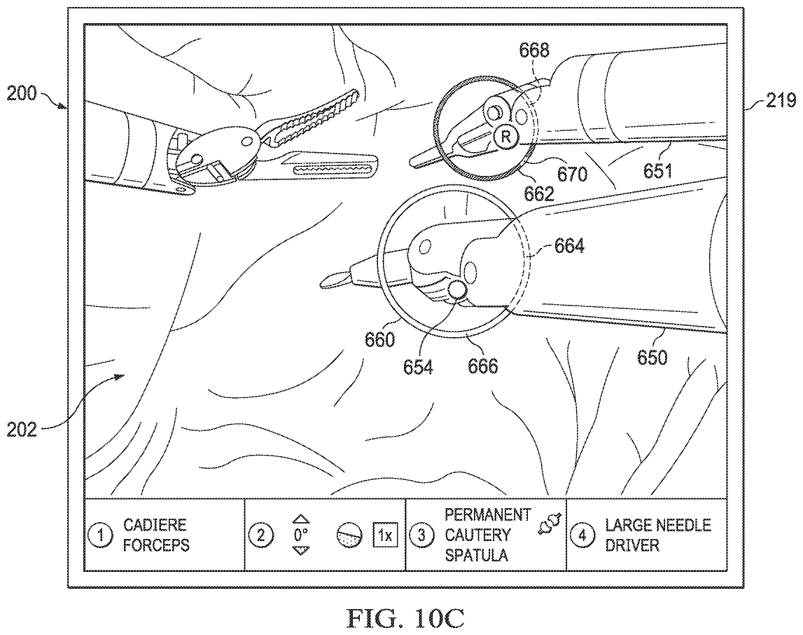

FIGS. 10A and 10B illustrate the graphical user interface 200 with a medical tool 650 visible in the field of view portion 202. In FIG. 10A a synthetic indicator 652 is rendered in the field of view portion 202 but appears to float above the tool 650 in the stereoscopic image. In FIG. 10B, a synthetic indicator 654 is rendered in the same position as indicator 652, but the rendering in FIG. 10AB creates the visual appearance that the synthetic indicator is embedded in the shaft of the tool 650. To create this visual appearance, an inner portion 656 of the synthetic indicator 654 is rendered with shading to demonstrate that the inner portion 656 is covered by or internal to the tool 650. An outer portion 658 of the synthetic indicator 654 is rendered with full opacity to indicate that the outer portion 658 is external to the tool 650.

FIG. 10C illustrate the graphical user interface 200 with the medical tool 650 and a medical tool 651 visible in the field of view portion 202. In FIG. 10C, a synthetic indicator 660 is rendered as a ring appearing to encircle the tool 650, and a synthetic indicator 662 is rendered as a ring appearing to encircle the tool 651. Using the depth aware blending techniques described above, a portion 664 of the synthetic indicator 660 that appears behind the tool 650 may be rendered in a different shading or color than a portion 666 of the synthetic indicator 660 that appears on top of the tool 650 and the surrounding tissue. Similarly, a portion 668 of the synthetic indicator 662 that appears behind the tool 651 may be rendered in a different shading or color than a portion 670 of the synthetic indicator 662 that appears on top of the tool 651.

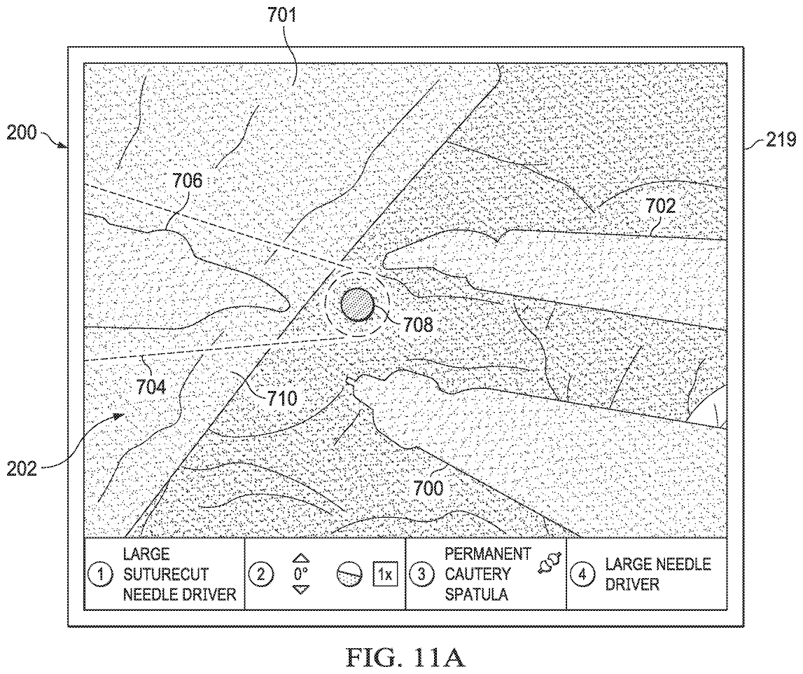

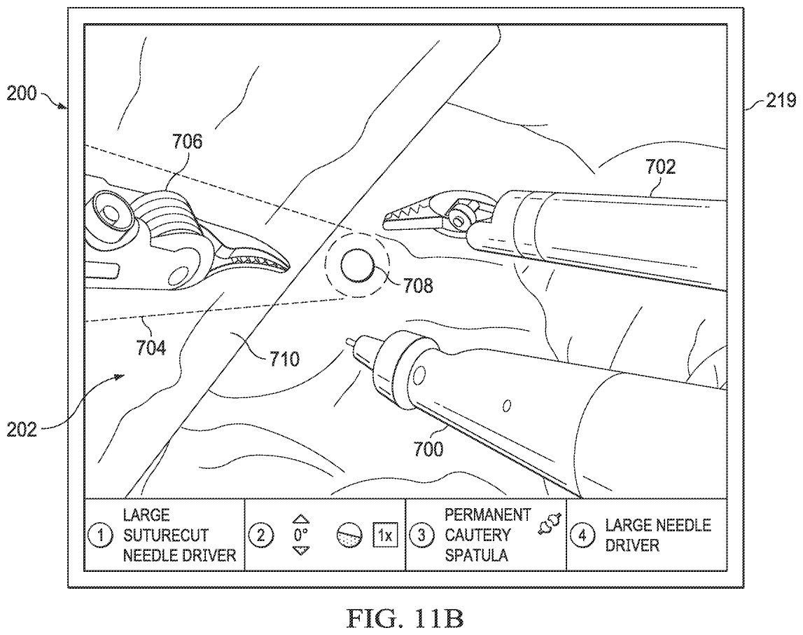

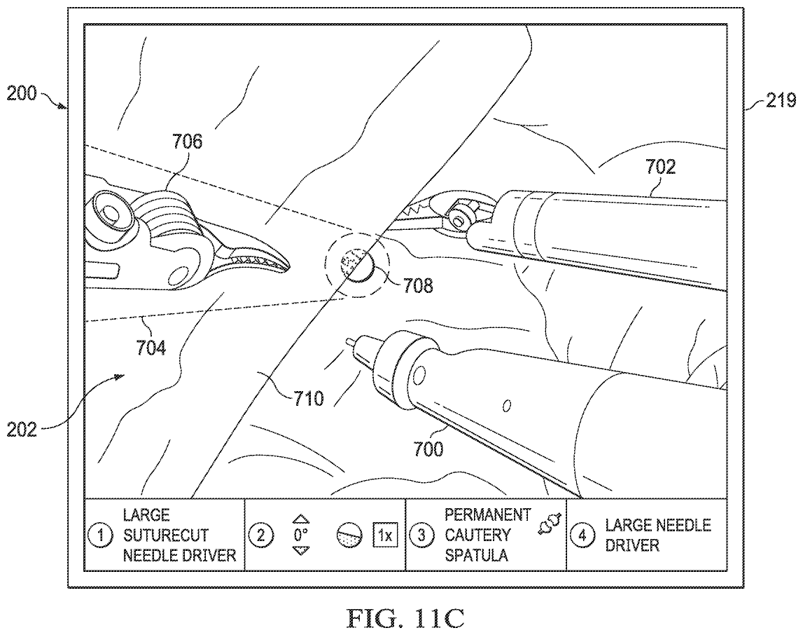

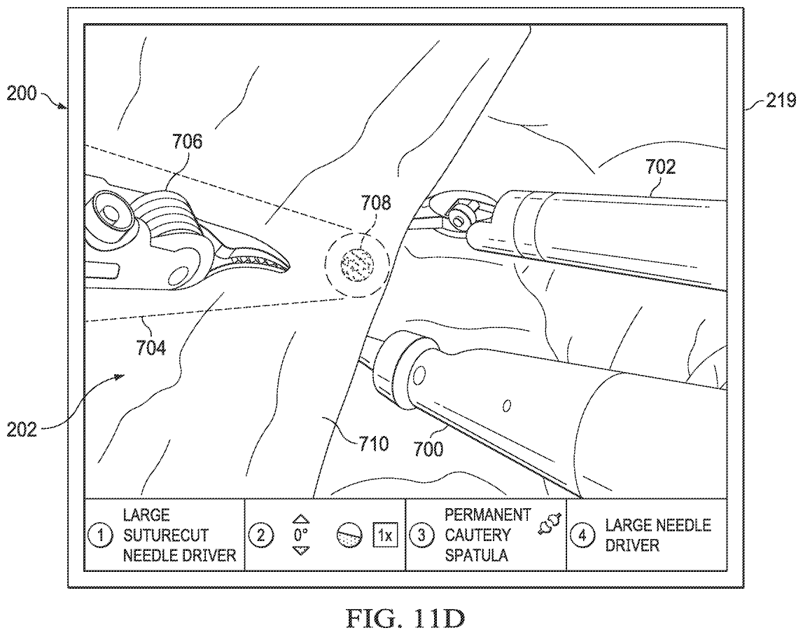

In some embodiments, the graphical user interface 200 may be used to display synthetic indicators for use in a guided tool change. The synthetic indicator may be rendered as a synthetic tube which serves as a path to guide the insertion of the new tool to a distal target mark. In some embodiments, all or portions of the synthetic tube may be occluded by tissue or other tools. FIGS. 11A-11D illustrate the graphical user interface 200 with different variations of the field of view portion 202. FIG. 11A illustrates a depth map 701 visualization of the field of view portion 202. A tool 700 and a tool 702 are visible in the field of view portion. A synthetic indicator 704 in the form of a synthetic tube may be provided to guide insertion of a tool 706 to a target mark 708. The depth map may indicate whether portions of the synthetic tube 704 or the target mark 708 are occluded by other structures. In FIG. 11B neither the synthetic tube 704 nor the target mark 708 are occluded, so the graphics for the synthetic tube 704 and the target mark 708 are presented without special visual properties or treatment. In FIG. 11C, tissue 710 obstructs a portion of the synthetic tube 704 and the target mark 708 so the occluded portions of the tube 704 and the mark 708 may have more translucent visual treatment than the nonoccluded portions to provide the viewer with information that the tool change path is partially obstructed by the tissue 710. In FIG. 11D, tissue 710 obstructs a greater portion of the synthetic tube 704 and fully obstructs the target mark 708. The occluded portions of the tube 704 and the mark 708 may have a more translucent visual treatment than the nonoccluded portions to provide the viewer with information that the tool change path is partially obstructed by the tissue 710. In these examples, opacity cues may provide an indication of the portions of the synthetic indicators that are occluded. In other examples, other visual properties may be modified with a two-stage rendering process (as described above) to modify color or texture properties that draw more attention to the occluded portions of the guided path. The occluded portion visual properties may be modified in a static or dynamic, time-varying manner. The depth map may also be used to answer geometric queries about the occluded state of the insertion path. One or more rays may be case that emanate from the tip of tool 706 along the insertion path direction toward the target mark. If an unobstructed insertion path is found that is closer to the target mark, the system may alert the viewer or adjust the synthetic indicator tube to be clear of the obstruction.

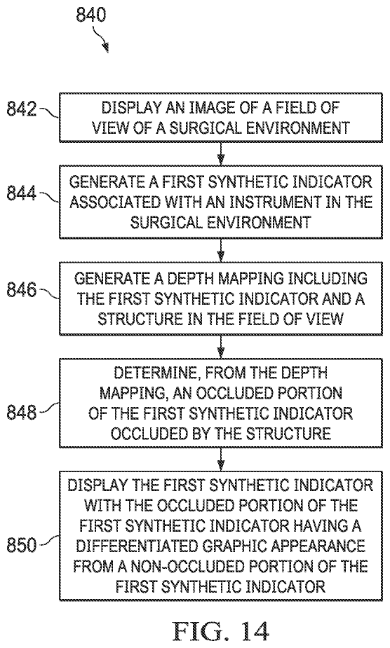

A method 840 for displaying partially occluded synthetic indicators is illustrated in the flowchart of FIG. 14. At a process 842, an image of a field of view (e.g., a field of view portion 202) of a surgical environment (e.g. environment 201) is displayed, for example on the display 35. At a process 844, a first synthetic indicator (e.g., synthetic mark 708) associated with an instrument (e.g., tool 706) in the surgical environment is generated. At a process 846, a depth mapping (e.g., depth map 701) including the first synthetic indicator and a structure in the field of view is generated. At a process 848, an occluded portion of the first synthetic indicator occluded by the structure is generated. At a process 850, the first synthetic indicator is displayed with the occluded portion of the first synthetic indicator having a differentiated graphic appearance from a non-occluded portion of the first synthetic indicator.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

Any alterations and further modifications to the described devices, systems, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately.

Various systems and portions of systems have been described in terms of their state in three-dimensional space. As used herein, the term “position” refers to the location of an object or a portion of an object in a three-dimensional space (e.g., three degrees of translational freedom along Cartesian X, Y, Z coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom).

Although some of the examples described herein refer to surgical procedures or instruments, or medical procedures and medical instruments, the techniques disclosed optionally apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system,” are analogous.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

1. A medical system comprising:
a display system; and
a control system, wherein the control system includes a processing unit including one or more processors, and wherein the processing unit is configured to:
display, on the display system, an image of a field of view of a surgical environment, wherein the image is generated by an imaging component;
generate a three-dimensional synthetic indicator for a position of an instrument outside of the field of view of the surgical environment; and
display the three-dimensional synthetic indicator with the image of the field of view of the surgical environment.

2. The medical system of claim 1 wherein the three-dimensional synthetic indicator includes an indicator body and an icon on the indicator body indicating a controlling manipulator arm.

3. The medical system of claim 2, wherein an orientation of the icon changes relative to the indicator body as the field of view changes.

4. The medical system of claim 1, wherein the three-dimensional synthetic indicator includes a three-dimensional directional indicator portion oriented toward the position of the instrument.

5. The medical system of claim 4 wherein generating the three-dimensional synthetic indicator includes determining a ray between a centerline of the imaging component and a point on the instrument.

6. The medical system of claim 1, wherein the three-dimensional synthetic indicator includes a height dimension, a width dimension, and a depth dimension.

7. The medical system of claim 1 wherein the three-dimensional synthetic indicator is pivotable relative to position of the instrument as the field of view changes.

8. The medical system of claim 1 wherein the three-dimensional synthetic indicator indicates instrument status or control mode.

9. The medical system of claim 1 wherein generating the three-dimensional synthetic indicator includes determining a depth range for structures in the field of view.

10. The medical system of claim 1 wherein the three-dimensional synthetic indicator morphs to indicate a distance between the three-dimensional synthetic indicator and instrument.

11. (canceled)

12. A medical system comprising:
a display system;
an input system including a first pedal and a second pedal, wherein the first pedal has a spatial relationship to the second pedal; and
a control system, wherein the control system includes a processing unit including one or more processors, and wherein the processing unit is configured to:
display, on the display system, an image of a field of view of a surgical environment, wherein the image is generated by an imaging component;
generate a first synthetic indicator indicating an engagement status of the first pedal;
generate a second synthetic indicator indicating an engagement status of the second pedal; and
display, on the display system, the first synthetic indicator relative to the second synthetic indicator based on the spatial relationship with the image of the field of view of the surgical environment.

13. The medical system of claim 12 wherein the first and second synthetic indicators are semicircular and positioned proximate to an instrument visible in the field of view and wherein the instrument is controlled by the input system.

14. The medical system of claim 12 wherein the first and second synthetic indicators are bars on a periphery of the image of the field of view.

15. The medical system of claim 12 wherein the engagement status of the first or second pedal is a pedal hover status.

16. The medical system of claim 12 wherein the engagement status of the first or second pedal is an activated status.

17. The medical system of claim 12 wherein the first and second synthetic indicators are badges positioned on an instrument clevis or shaft.

18. The medical system of claim 17 wherein the badges change size with an endoscopic zoom and wherein the change of size is subject to maximum and minimum thresholds.

19. The medical system of claim 17 wherein a badge 3D orientation moves with clevis or shaft.

20. The medical system of claim 17 wherein the badges face an endoscope tip and appear upright.

21. The medical system of claim 12 wherein the engagement status of the first or second pedal is detected by a sensor system.

22-31. (canceled)
